(19) United States
(12) Patent Application Publication
(10) Pub. No.: US 2011/0294995 A1
(43) Pub. Date: Dec. 1, 2011

(54) Functionalized nanoparticles and other particles and methods for making and using same

(76) Inventors: Qun Huo, Fargo, ND (US); James G. Worden, Fargo, ND (US)

(21) Appl. No.: 10/793,592

(22) Filed: Mar. 4, 2004

Related U.S. Application Data
(60) Provisional application No. 60/451,995, filed on Mar. 5, 2003.
(60) Provisional application No. 60/492,386, filed on Aug. 4, 2003.

(51) Int. Cl. C07H 21/02; C07H 21/04; B82Y 99/00

(52) U.S. Cl. 536/23.1; 977/773

(57) ABSTRACT

Disclosed are nanoparticles which include a nanoparticle core having a core diameter of greater than 5 nm and a single functional moiety bonded to the nanoparticle core. Also disclosed are nanoparticles which include a nanoparticle core having a core diameter of greater than 1.4 nm and a single functional moiety bonded to the nanoparticle core, wherein the single functional moiety does not contain a nucleic acid molecule that includes 100 or more nucleotides. A method for preparing a functionalized nanoparticle is also disclosed. The method includes providing a nanoparticle core and providing a functionalizing moiety that includes a functional group and a reactive group. The functional group is attached to a substrate surface, and the reactive group is capable of bonding to the nanoparticle core's surface. The method further includes contacting the nanoparticle core's surface with the functionalizing moiety under conditions effective to bind the functionalizing moiety's reactive group and the nanoparticle core's surface together.

The present application claims the benefit of U.S. Provisional Patent Application Ser. No. 60/451,995, filed Mar. 5, 2003, and U.S. Provisional Patent Application Ser. No. 60/492,386, filed Aug. 4, 2003, each of which provisional patent applications is hereby incorporated by reference.

The present invention was made with the support of NSF/EPSCoR Grant Nos. EPS-0132289 and EPS-9874802 and with support of National Science Foundation Grant No. DMR 0239424. The Federal Government may have certain rights in this invention.

### FIELD OF THE INVENTION

The present invention is directed, generally, to nanoparticles and, more particularly, to nanoparticles which include a metal core and a single functional moiety bound thereto, and to methods for making and using such nanoparticles.

### BACKGROUND OF THE INVENTION

The synthesis and study of gold nanoparticles is a major area of current nanoparticle and nanomaterial research. For example, see Schmid, Clusters and Colloids: From Theory to Applications, New York: VCH (1994); Templeton et al., Acc. Chem. Res., 33:27ff (2000); and Schmid et al., Chem. Soc. Rev., 28:179ff (1999). Gold nanoparticles exhibit dimension-dependent properties in the nanoscale range, such as quantized charging effect, surface plasma resonance absorption, surface-enhanced Raman scattering, luminescence, fluorescence, and other unique properties. Gold nanoclusters are potential candidates for many applications such as nanoelectronics, nanodevices, biosensor, and chemosensor development. However, a significant challenge present in current nanoparticle research is how to prepare gold nanoparticles with pre-defined chemical structures and controlled number of functionalities. Monolayer-protected gold nanoparticles are commonly prepared by the reported Schiffrin and/or place-exchange reaction. Due to the nature of these two reactions, the obtained nanoparticles often contain multiple unknown numbers of surface functional groups. In order to control the nanomaterial and nanodevice structures built from nanoparticles, it would be highly desirable if the chemical structure and functionality of the nanoparticles could be controlled. The present invention is directed, in part, to addressing this need.

### SUMMARY OF THE INVENTION

The present invention relates to a nanoparticle which includes a nanoparticle core having a core diameter of greater than 5 nm and a single functional moiety bonded to the nanoparticle core.

The present invention also relates to a nanoparticle which includes a nanoparticle core having a core diameter of greater than 1.4 nm and a single functional moiety bonded to the nanoparticle core, wherein the single functional moiety does not contain a nucleic acid molecule that includes 100 or more nucleotides.

The present invention also relates to a method for preparing a functionalized nanoparticle. The method includes providing a nanoparticle core and providing a functionalizing moiety that includes a functional group and a reactive group. The functional group is attached to a substrate surface, and the reactive group is capable of bonding to the nanoparticle core's surface. The method further includes contacting the nanoparticle core's surface with the functionalizing moiety under conditions effective to bind the functionalizing moiety's reactive group and the nanoparticle core's surface together.

### BRIEF DESCRIPTION OF THE DRAWINGS

FIG. 1 shows a synthetic scheme for preparing various nanoparticles according to the present invention.

FIGS. 2A and 2B are TEM images of various nanoparticles according to the present invention.

FIG. 3 is TEM image of 1,7-diaminoheptane coupled 5% place exchange product.

FIG. 4 is a general synthetic scheme for preparing various nanoparticles according to the present invention.

FIG. 5 is a graphic depicting a nanoparticle of the present invention with a single copy of surface monofunctional group.

FIG. 6 is a synthetic scheme for preparing nanoparticles according to the present invention.

FIG. 7A is a graphic representation of a pair of QCA double-dot cells. FIG. 7B is a graphic representation of a simple inverter logic device based on QCA double-dot cell.

FIG. 8A is a graphic depicting a nanoparticle dimer of the present invention which can be used as a QCA double-dot cell. FIG. 8B is a graphic depicting a nanoparticle trimer of the present invention.

FIG. 9 is a graphic depicting a nanoparticle in accordance with the present invention containing two chain-extended glycine amino acids.

FIG. 10A is a graphic representation of a QCA line. FIG. 10B is a graphic of a nanoparticle in accordance with the present invention that mimics a QCA line.

FIG. 11A is synthetic scheme for preparing peptide-containing nanoparticles according to the present invention. FIG. 11B is a graphic illustrating one orientation of peptide-containing nanoparticles according to the present invention at an air-water interface. FIG. 11C is a graphic illustrating two different packing configurations for peptide-containing nanoparticles according to the present invention at an air-water interface.

### DETAILED DESCRIPTION OF THE INVENTION

The present invention, in one aspect thereof, relates to a nanoparticle which includes a nanoparticle core having a core diameter of greater than 5 nm and a single functional moiety bonded to the nanoparticle core.

The present invention, in another aspect thereof, relates to a nanoparticle which includes a nanoparticle core having a core diameter of greater than 1.4 nm and which further includes a single functional moiety bonded to the nanoparticle core, wherein the single functional moiety does not contain a nucleic acid molecule comprising 100 or more nucleotides.

The nature of the bond between the functional moiety and the nanoparticle core is not particularly critical to the practice of the present invention. Illustratively, the functional moiety and the nanoparticle core can be bonded together via a covalent interaction, via an ionic interaction, via a hydrogen bond interaction, via a pi-pi orbital interaction, via a van der Waals interaction, or via combinations thereof.

The nanoparticle core can be a metallic nanoparticle core, i.e., a nanoparticle core which is made substantially exclusively of metal atoms (e.g., greater than about 60% by weight, greater than about 70% by weight, greater than about 80% by weight, greater than about 90% by weight, greater than about 95% by weight, and/or greater than about 98% by weight of metal atoms). Suitable metallic nanoparticle cores include those in which substantially all (e.g., greater than about 60% by weight, greater than about 70% by weight, greater than about 80% by weight, greater than about 90% by weight, greater than about 95% by weight, and/or greater than about 98% by weight) of the metal atoms are of the same type (i.e., have the same atomic number), for example, as in the case where substantially all of the metal atoms are gold atoms, as in the case where substantially all of the metal atoms are silver atoms, as in the case where substantially all of the metal atoms are platinum atoms, as in the case where substantially all of the metal atoms are palladium atoms, or as in the case where substantially all of the metal atoms are copper atoms. Other metal atoms which can be contained in the nanoparticle core and/or from which the nanoparticle core can be made include, for example, Li, Na, K, Rb, Cs, Be, Mg, Ca, Sr, Ba, Ra, Sc, Y, Ti, Zr, Hf, V, Nb, Ta, Cr, Mo, W, Mn, Tc, Re, Fe, Ru, Os, Co, Rh, Ir, Ni, Pd, Zn, Cd, Hg, Al, Ga, In, Tl, Ge, Sn, Pb, Sb, Bi, Po, La, Ce, Pr, Nd, Pm, Sm, Eu, Gd, Tb, Dy, Ho, Er, Tm, Yb, Lu, Ac, Th, Pa, U, and/or Np. Other suitable metallic nanoparticle cores include those which include two, three, four, or more different types of metal atoms (i.e., two, three, four, or more metal atoms having different atomic numbers). Ceramic, glass, glass-ceramic, or other types of substantially inorganic nanoparticles (such as semiconductor nanocrystals, e.g., semiconductor nanocrystals containing ZnS, CdSe, ZnSe, ZnTe, and other semiconductor nanocrystals containing a chalcogen; and metal oxide nanocrystals, e.g., metal oxide nanocrystals containing ZrO₂, CuO, CuO₂, Gd₂O₃, Y₂O₃, etc.) can also be employed as the nanoparticle cores. The nanoparticle core can include atoms that are traditionally viewed as non-metallic atoms (such as halogen atoms, oxygen, sulfur, selenium, tellurium, nitrogen, phosphorus, arsenic, carbon, silicon, germanium, and boron), or the nanoparticle core can be substantially free from one, more than one, or all of these types of atoms, for example, as in the case where the nanoparticle core is substantially free of carbon. The atoms which make up the nanoparticle core can be bound together via any suitable type or types of bonds, such as metallic bonds, covalent bonds, ionic bonds, etc.

The nanoparticle core can be of any suitable shape, such as spherical, substantially spherical, ellipsoidal, polyhedral (e.g., cube-shaped, tetrahedron-shaped, octahedron-shaped, etc.), flat (e.g., disk-shaped), one-dimensional (e.g., needle-shaped), and the like.

The nanoparticle core can be of any suitable size.

For example, in cases where the nanoparticle includes a nanoparticle core having a core diameter of greater than 5 nm and a single functional moiety bonded to the nanoparticle core, the nanoparticle core can have a diameter of greater than about 5.5 nm, such as greater than about 6 nm, greater than about 7 nm, greater than about 8 nm, greater than about 9 nm, and/or greater than about 10 nm. As further illustration, the nanoparticle core can have a diameter of from greater than 5 nm to about 1000 nm, from about 5.5 nm to about 1000 nm, from about 6 nm to about 1000 nm, from about 7 nm to about 1000 nm, from about 8 nm to about 1000 nm, from about 9 nm to about 1000 nm, from about 10 nm to about 1000 nm, from about 20 nm to about 1000 nm, from greater than 5 nm to about 500 nm, from about 5.5 nm to about 500 nm, from about 6 nm to about 500 nm, from about 7 nm to about 500 nm, from about 8 nm to about 500 nm, from about 9 nm to about 500 nm, from about 10 nm to about 500 nm, from about 20 nm to about 500 nm, from greater than 5 nm to about 200 nm, from about 5.5 nm to about 200 nm, from about 6 nm to about 200 nm, from about 7 nm to about 200 nm, from about 8 nm to about 200 nm, from about 9 nm to about 200 nm, from about 10 nm to about 200 nm, from about 20 nm to about 200 nm, from greater than 5 nm to about 100 nm, from about 5.5 nm to about 100 nm, from about 6 nm to about 100 nm, from about 7 nm to about 100 nm, from about 8 nm to about 100 nm, from about 9 nm to about 100 nm, from about 10 nm to about 100 nm, from about 20 nm to about 100 nm, from greater than 5 nm to about 50 nm, from about 5.5 nm to about 50 nm, from about 6 nm to about 50 nm, from about 7 nm to about 50 nm, from about 8 nm to about 50 nm, from about 9 nm to about 50 nm, from about 10 nm to about 50 nm, from about 20 nm to about 50 nm, from greater than 5 nm to about 25 nm, from about 5.5 nm to about 25 nm, from about 6 nm to about 25 nm, from about 7 nm to about 25 nm, from about 8 nm to about 25 nm, from about 9 nm to about 25 nm, from about 10 nm to about 25 nm, from about 20 nm to about 25 nm, from greater than 5 nm to about 20 nm, from about 5.5 nm to about 20 nm, from about 6 nm to about 20 nm, from about 7 nm to about 20 nm, from about 8 nm to about 20 nm, from about 9 nm to about 20 nm, from about 10 nm to about 20 nm, from greater than 5 nm to about 15 nm, from about 5.5 nm to about 15 nm, from about 6 nm to about 15 nm, from about 7 nm to about 15 nm, from about 8 nm to about 15 nm, from about 9 nm to about 15 nm, from about 10 nm to about 15 nm, from greater than 5 nm to about 10 nm, from about 5.5 nm to about 10 nm, from about 6 nm to about 10 nm, from about 7 nm to about 10 nm, from about 8 nm to about 10 nm, and/or from about 9 nm to about 10 nm.

In cases where the nanoparticle includes a nanoparticle core having a core diameter of greater than 1.4 nm and which further includes a single functional moiety bonded to the nanoparticle core, wherein the single functional moiety does not contain a nucleic acid molecule comprising 100 or more nucleotides, the nanoparticle core can have a diameter of greater than about 1.5 nm, greater than about 1.6 nm, greater than about 1.7 nm, greater than about 1.8 nm, greater than about 1.9 nm, and/or greater than about 2 nm. As further illustration, the nanoparticle core can have a diameter of from greater than 1.4 nm to about 1000 nm, such as from about 1.5 nm to about 1000 nm, from about 1.7 nm to about 1000 nm, from about 2 nm to about 1000 nm, from about 2.5 nm to about 1000 nm, from about 3 nm to about 1000 nm, from about 4 nm to about 1000 nm, from about 5 nm to about 1000 nm, from about 7 nm to about 1000 nm, from greater than 1.4 nm to about 500 nm, from about 1.5 nm to about 500 nm, from about 1.7 nm to about 500 nm, from about 2 nm to about 500 nm, from about 2.5 nm to about 500 nm, from about 3 nm to about 500 nm, from about 4 nm to about 500 nm, from about 5 nm to about 1000 nm, from about 7 nm to about 500 nm, from greater than 1.4 nm to about 200 nm, from about 1.5 nm to about 200 nm, from about 1.7 nm to about 200 nm, from about 2 nm to about 200 nm, from about 2.5 nm to about 200 nm, from about 3 nm to about 200 nm, from about 4 nm to about 200 nm, from about 5 nm to about 200 nm, from about 7 nm to about 200 nm, from greater than 1.4 nm to about 100 nm, from about 1.5 nm to about 100 nm, from about 1.7 nm to about 100 nm, from about 2 nm to about 100 nm, from about 2.5 nm to about 100 nm, from about 3 nm to about 100 nm, from about 4 nm to about 100 nm, from about 5 nm to about 100 nm, from about 7 nm to about 100 nm, from greater than 1.4 nm to about 50 nm, from about 1.5 nm to about 50 nm, from about 1.7 nm to about 50 nm, from about 2 nm to about 50 nm, from about 2.5 nm to about 50 nm, from about 3 nm to about 50 nm, from about 4 nm to about 50 nm, from about 5 nm to about 50 nm, from about 7 nm to about 50 nm, from greater than 1.4 nm to about 25 nm, from about 1.5 nm to about 25 nm, from about 1.7 nm to about 25 nm, from about 2 nm to about 25 nm, from about 2.5 nm to about 25 nm, from about 3 nm to about 25 nm, from about 4 nm to about 25 nm, from about 5 nm to about 25 nm, from about 7 nm to about 25 nm, from greater than 1.4 nm to about 10 nm, from about 1.5 nm to about 10 nm, from about 1.7 nm to about 10 nm, from about 2 nm to about 10 nm, from about 2.5 nm to about 10 nm, from about 3 nm to about 10 nm, from about 4 nm to about 10 nm, from about 5 nm to about 10 nm, and/or from about 7 nm to about 10 nm.

In either case, when the nanoparticle core has a shape other than that of a sphere, “diameter” is meant to refer the diameter of a hypothetical sphere having the same volume as that of the nanoparticle core. When the nanoparticle is present in a collection of nanoparticles and the of nanoparticles in the collection have cores of varying sizes, “diameter” is meant to refer the mean diameter of the nanoparticle cores present in the collection of nanoparticles.

As used herein, a functional moiety is meant to include any moiety that contains one functional group, two functional groups, three functional groups, four functional groups, or more than four functional groups.

Functional groups are meant to include any and all groups that are capable of being exploited in a chemical reaction and/or which are capable of interacting with other functional groups (e.g., undergo hydrogen bonding), such as groups containing halogen atoms, oxygen atoms, sulfur atoms, selenium atoms, nitrogen atoms, and/or phosphorus atoms, example of which include hydroxyl groups, carboxylic acid groups (which is meant to include salts thereof), ester groups, aldehyde groups, amine groups (which is meant to include primary, secondary, and tertiary amine groups), amide groups, thiol groups (which is meant to include substituted and unsubstituted thiols), and the like. Other functional groups can also be employed, such as those which contain a C—H bond that is particularly reactive, as in the case where the functional moiety contains a —CH(C₆H₅)₃ functional group.

Functional groups also include those which contain one or more amino acids, which are meant to include α-amino acids, β-amino acids, naturally occurring amino acids, non-naturally occurring amino acids, or combinations thereof. For example, suitable functional groups include those which contain more than 10 peptide bonds as well as those which contain fewer than 10 peptide bonds, those which contain fewer than 9 peptide bonds, those which contain fewer than 8 peptide bonds, those which contain fewer than 7 peptide bonds, those which contain fewer than 6 peptide bonds, those which contain fewer than 5 peptide bonds, those which contain fewer than 4 peptide bonds, those which contain fewer than 3 peptide bonds, those which contain fewer than 2 peptide bonds, and/or those which contain no peptide bonds.

In cases where the nanoparticle includes a nanoparticle core having a core diameter of greater than 5 nm and a single functional moiety bonded to the nanoparticle core, suitable functional groups also include those which contain no nucleic acid molecules as well as those which contain nucleic acid molecules. Functional moieties suitable for use in the nanoparticles of this aspect of the present invention include those which contain nucleic acid molecules comprising fewer than about 100 nucleotides, fewer than 100 nucleotides, fewer than about 80 nucleotides, fewer than 80 nucleotides fewer than about 50 nucleotides, and/or fewer than 50 nucleotides. Other functional moieties suitable for use in the nanoparticles of this aspect of the present invention include those which contain nucleic acid molecules comprising about 100 or more nucleotides, comprising 100 or more nucleotides, comprising about 80 or more nucleotides, comprising 80 or more nucleotides, comprising about 50 or more nucleotides, and/or comprising 50 or more nucleotides.

In cases where the nanoparticle includes a nanoparticle core having a core diameter of greater than 1.4 nm and a single functional moiety bonded to the nanoparticle core, wherein the single functional moiety does not contain a nucleic acid molecule comprising 100 or more nucleotides, suitable functional groups also include those which contain no nucleic acid molecules as well as those which contain nucleic acid molecules comprising fewer than 100 nucleotides.

In either case, examples of nucleic acid molecules comprising fewer than 100 nucleotides include those comprising fewer than about 95 nucleotides and/or fewer than 95 nucleotides, such as fewer than about 90 nucleotides, fewer than 90 nucleotides, fewer than about 85 nucleotides, fewer than 85 nucleotides, fewer than about 80 nucleotides, fewer than 80 nucleotides, fewer than about 75 nucleotides, fewer than 75 nucleotides, fewer than about 70 nucleotides, fewer than 70 nucleotides, fewer than about 65 nucleotides, fewer than 65 nucleotides, fewer than about 60 nucleotides, fewer than 60 nucleotides, fewer than about 55 nucleotides, fewer than 55 nucleotides, fewer than about 50 nucleotides, fewer than 50 nucleotides, fewer than about 45 nucleotides, fewer than 45 nucleotides, fewer than about 40 nucleotides, fewer than 40 nucleotides, fewer than about 35 nucleotides, fewer than 35 nucleotides, fewer than about 30 nucleotides, fewer than 30 nucleotides, fewer than about 25 nucleotides, fewer than 25 nucleotides, fewer than about 20 nucleotides, fewer than 20 nucleotides, fewer than about 10 nucleotides, fewer than 10 nucleotides, fewer than about 5 nucleotides, fewer than 5 nucleotides, fewer than about 2 nucleotides, and/or fewer than 2 nucleotides.

As used herein, “nucleic acid molecules” are meant to include, for example, oligonucleotides, DNA molecules, and RNA molecules, the nucleotides of which can be naturally occurring, non-naturally occurring, or combinations thereof.

As indicated above, suitable functional moieties include those which contain functional groups in their unreacted form (e.g., as free hydroxyl groups, free carboxylic acid or carboxylate salt groups, free primary amine or secondary groups, free thiol groups, etc.). Functional moieties are also meant to include moieties which contain functional groups that have been incorporated into a reaction product between an unreacted functional group and a reactive group of a reactive species. For example, the functional group can be an ester (i.e., the product of a free (unreacted) hydroxyl functional group and a reactive species containing a free carboxylic acid or carboxylate salt group; the product of a free (unreacted) carboxylic acid or carboxylate salt functional group and a reactive species containing a hydroxyl group; etc.). The reactive species can be free of other functional groups (i.e., other than the one which is involved in the reaction with the functional moiety's functional group), for example, as in the case where the reactive species is a non-functionalized nanoparticle and where the nanoparticle of the present invention has the formula (NC)-R-Q-R′-(NFNP), where NC represents the subject nanoparticle's nanoparticle core, —R-Q-R′-(NFNP) represents the subject nanoparticle's functional moiety, R and R′ represent the same or different linking groups (e.g., alkylene moieties), Q represents a moiety resulting from the reaction between the unreacted functional group and the reactive group of a reactive species, and NFNP represents a non-functionalized nanoparticle. Alternatively, the reactive species can include other functional groups (i.e., in addition to the one which is involved in the reaction with the functional moiety's functional group), for example, as in the case (i) where the reactive species is a functionalized nanoparticle and where the nanoparticle of the present invention has the formula (NC)-R-Q-R′-(FNP), where NC represents the subject nanoparticle's nanoparticle core, —R-Q-R′-(FNP) represents the subject nanoparticle's functional moiety, each R represents the same or a different linking group (e.g., alkylene moieties), Q represents a moiety resulting from the reaction between the unreacted functional group and the reactive group of a reactive species, and FNP represents a functionalized nanoparticle; (ii) where the reactive species is a polypeptide and where the nanoparticle of the present invention has the formula (NC)-R-Q-R′-(PP), where NC represents the subject nanoparticle's nanoparticle core, —R-Q-R′-(PP) represents the subject nanoparticle's functional moiety, R and R′ represent the same or different linking groups (e.g., alkylene moieties), Q represents a moiety resulting from the reaction between the unreacted functional group and the reactive group of a reactive species, and PP represents a polypeptide (e.g., an antibody, a protein, etc.); and (iii) where the reactive species is a nucleic acid molecule and where the nanoparticle of the present invention has the formula (NC)-R-Q-R′-(NA), where NC represents the subject nanoparticle's nanoparticle core, —R-Q-R′-(NA) represents the subject nanoparticle's functional moiety, R and R′ represent the same or different linking groups (e.g., alkylene moieties), Q represents a moiety resulting from the reaction between the unreacted functional group and the reactive group of a reactive species, and NA represents a nucleic acid (e.g., an RNA, a DNA, an oligonucleotide, etc.).

“Functional moieties”, as used herein, are also meant to include cells or portions thereof; subcellular organelles; viruses; bacteria; and the like.

As further illustration, the nanoparticles of the present invention include, for example, nanoparticles having the formula (NC)-R-T, where (NC) represents a nanoparticle core, such as one of the nanoparticle cores discussed hereinabove; R-T represents a functional moiety; R represents a linking group such as a substituted or unsubstituted alkylene moiety (e.g., a moiety having the formula —(CH₂)ₙ, where n an integer from 1 to about 24, such as from about 3 to about 12, from about 4 to about 8, and/or equal to 4, 5, 6, 7, or 8); and T represents a moiety comprising (i) at least one free functional group (such as hydroxyl, carboxylic acid or salt, primary amine, secondary amine, thiol, etc.) and/or (ii) at least one polypeptide, at least one nucleic acid molecule, and/or at least one nanoparticle.

As discussed above, the functional moiety can be a moiety that contains one functional group, two functional groups, three functional groups, four functional groups, or more than four functional groups. Illustratively, the functional moiety can contain a single hydroxyl group, a single carboxylic acid or carboxylate salt group, a single amine group, a single thiol group, etc.; or it can contain two or more of such groups, for example, as in the case where the functional moiety contains at least one carboxylic acid or carboxylate salt group and at least one amine group.

Also as discussed above, the subject nanoparticle includes a single (i.e., one and only one) functional moiety bonded to the nanoparticle core.

“Functional moieties”, as used herein, are not meant to include moieties which contain one or more heteroatoms where each of the one or more heteroatoms is bonded directly to the nanoparticle. For example, a an alkylthio moiety whose sulfur atom is bonded to the nanoparticle is not to be construed as being a “functional moiety” for the purposes of the present invention. As further illustration, a moiety having the formula —S—CH(R)—S— is to be construed as being a functional moiety (i) if one of the sulfur atoms is not bonded to the nanoparticle and/or (ii) if the R group contains one or more heteroatoms or other functional groups; otherwise the —S—CH(R)—S— moiety is not to be construed to be a functional moiety.

As discussed above, “functional moieties” are not meant to include moieties which contain one or more heteroatoms where each of the one or more heteroatoms is bonded to the nanoparticle. For the purposes of the present invention, moieties which contain one or more heteroatoms where each of the one or more heteroatoms is bonded directly to the nanoparticle are termed “non-functional moieties”. The nanoparticles of the present invention can further include (i.e., in addition to the nanoparticle core and the single functional moiety) one or more “non-functional moieties” bonded to the nanoparticle core. Examples of such “non-functional moieties” include non-functional phosphine moieties (e.g., non-functional triphenylphosphine moieties and other non-functional triarylphosphine moieties) and non-functional thiol moieties (e.g., non-functional alkylthio moieties and non-functional arylthio moieties). Combinations of these and other non-functional moieties can be employed.

Illustratively, the nanoparticles of the present invention include those which can be represented by the formula:

(NFM)ₓ-(NC)-(FM)

where NC represents a nanoparticle core; FM represents a functional moiety, such as one of the ones described above; NFM represents a non-functional moiety (e.g., an alkylthio group where the alkylthio group's sulfur atom is bonded to the nanoparticle core); and x represents 0 or an integer from 1 to Q where Q is the maximum number of NFM that can be bound to the nanoparticle core, such as the number of NFM needed to form a protective monolayer around the nanoparticle core). For example, FM can comprise a nucleic acid molecule, such as an oligonucleotide, a DNA molecule, or an RNA molecule; a polypeptide, such as an antibody or a protein); a nanoparticle; a cell or a portion thereof; a subcellular organelle; a virus; a bacteria; a reactive functional group, such as a free hydroxy group, a free carboxylic acid or carboxylate salt group, a free thiol group, a free amine group, etc.; a protected functional group, such as a protected hydroxy group (e.g., protected with an acetyl or another alkylcarbonyl group or protected as an ether, such as a t-butyl ether, a trityl ether, or a trialkylsilyl ether), a protected carboxylic acid or carboxylate salt group (e.g., protected as an ester, such as a benzyl ester, a t-butyl ester, a methyl ester, etc.), a protected thiol group (e.g., protected with an acetyl or another alkylcarbonyl group or protected as a thioether, such as a t-butyl thioether, a trityl thioether, or a trialkylsilyl thioether), a protected amine group (e.g., protected with an alkylcarbonyl or a alkoxycarbonyl group, such as a benzyloxycarbonyl group, a t-butoxycarbonyl group, or a 2-biphenyl-2-propoxycarbonyl group), etc.; or combinations thereof. NC can have the formula Mₙ where Mₙ represents a cluster of metal atoms (e.g., metal atoms selected from the group consisting of Au, Ag, Pt, Pd, Cu, and combinations thereof). Illustratively, in cases where the nanoparticle includes a nanoparticle core having a core diameter of greater than 5 nm and a single functional moiety bonded to the nanoparticle core, n can be an integer greater than 2700 but less than about 10⁹ atoms, such as between about 2800 atoms and about 10⁹ atoms, between about 3000 atoms and about 10⁹ atoms, between about 3500 atoms and about 10⁹ atoms, between about 4000 atoms and about 10⁹ atoms, between about 4500 atoms and about 10⁹ atoms, between about 5000 atoms and about 10⁹ atoms, between about 10000 atoms and about 10⁹ atoms, greater than 2700 but less than about 10⁸ atoms, between about 2800 atoms and about 10⁸ atoms, between about 3000 atoms and about 10⁸ atoms, between about 3500 atoms and about 10⁸ atoms, between about 4000 atoms and about 10⁸ atoms, between about 4500 atoms and about 10⁸ atoms, between about 5000 atoms and about 10⁸ atoms, between about 10000 atoms and about 10⁸ atoms, greater than 2700 but less than about 10⁸ atoms, between about 2800 atoms and about 10⁷ atoms, between about 3000 atoms and about 10⁷ atoms, between about 3500 atoms and about 10⁷ atoms, between about 4000 atoms and about 10⁷ atoms, between about 4500 atoms and about 10⁷ atoms, between about 5000 atoms and about 10⁷ atoms, between about 10000 atoms and about 10⁷ atoms, greater than 2700 but less than about 10⁶ atoms, between about 2800 atoms and about 10⁶ atoms, between about 3000 atoms and about 10⁶ atoms, between about 3500 atoms and about 10⁶ atoms, between about 4000 atoms and about 10⁶ atoms, between about 4500 atoms and about 10⁶ atoms, between about 5000 atoms and about 10⁶ atoms, between about 10000 atoms and about 10⁶ atoms, greater than 2700 but less than about 10⁵ atoms, between about 2800 atoms and about 10⁵ atoms, between about 3000 atoms and about 10⁵ atoms, between about 3500 atoms and about 10⁵ atoms, between about 4000 atoms and about 10⁵ atoms, between about 4500 atoms and about 10⁵ atoms, between about 5000 atoms and about 10⁵ atoms, between about 10000 atoms and about 10⁵ atoms, greater than 2700 but less than about 10⁵ atoms, between about 2800 atoms and about 10⁵ atoms, between about 3000 atoms and about 10⁵ atoms, between about 3500 atoms and about 10⁵ atoms, between about 4000 atoms and about 10⁵ atoms, between about 4500 atoms and about 10⁵ atoms, between about 5000 atoms and about 10⁵ atoms, between about 10000 atoms and about 10⁵ atoms, greater than 2700 but less than about 10⁴ atoms, between about 2800 atoms and about 10⁴ atoms, between about 3000 atoms and about 10⁴ atoms, between about 3500 atoms and about 10⁴ atoms, between about 4000 atoms and about 10⁴ atoms, between about 4500 atoms and about 10⁴ atoms, and/or between about 5000 atoms and about 10⁴ atoms. As further illustration, in cases where the nanoparticle includes a nanoparticle core having a core diameter of greater than 1.4 nm and a single functional moiety bonded to the nanoparticle core, wherein the single functional moiety does not contain a nucleic acid molecule comprising 100 or more nucleotides, n can be greater than 70 but less than about 10⁹ atoms, such as between about 75 atoms and about 10⁹ atoms, between about 80 atoms and about 10⁹ atoms, between about 90 atoms and about 10⁹ atoms, between about 100 atoms and about 10⁹ atoms, between about 200 atoms and about 10⁹ atoms, between about 500 atoms and about 10⁹ atoms, between about 1000 atoms and about 10⁹ atoms, between about 2000 atoms and about 10⁹ atoms, between about 5000 atoms and about 10⁹ atoms, between about 10000 atoms and about 10⁹ atoms, greater than 70 but less than about 10⁸ atoms, between about 75 atoms and about 10⁸ atoms, between about 80 atoms and about 10⁸ atoms, between about 90 atoms and about 10⁸ atoms, between about 100 atoms and about 10⁸ atoms, between about 200 atoms and about 10⁸ atoms, between about 500 atoms and about 10⁸ atoms, between about 1000 atoms and about 10⁸ atoms, between about 2000 atoms and about 10⁸ atoms, between about 5000 atoms and about 10⁸ atoms, between about 10000 atoms and about 10⁸ atoms, greater than 70 but less than about 10⁷ atoms, between about 75 atoms and about 10⁷ atoms, between about 80 atoms and about 10⁷ atoms, between about 90 atoms and about 10⁷ atoms, between about 100 atoms and about 10⁷ atoms, between about 200 atoms and about 10⁷ atoms, between about 500 atoms and about 10⁷ atoms, between about 1000 atoms and about 10⁷ atoms, between about 2000 atoms and about 10⁷ atoms, between about 5000 atoms and about 10⁷ atoms, between about 10000 atoms and about 10⁷ atoms, greater than 70 but less than about 10⁶ atoms, between about 75 atoms and about 10⁶ atoms, between about 80 atoms and about 10⁶ atoms, between about 90 atoms and about 10⁶ atoms, between about 100 atoms and about 10⁶ atoms, between about 200 atoms and about 10⁶ atoms, between about 500 atoms and about 10⁶ atoms, between about 1000 atoms and about 10⁶ atoms, between about 2000 atoms and about 10⁶ atoms, between about 5000 atoms and about 10⁶ atoms, between about 10000 atoms and about 10⁶ atoms, greater than 70 but less than about 10⁵ atoms, between about 75 atoms and about 10⁵ atoms, between about 80 atoms and about 10⁵ atoms, between about 90 atoms and about 10⁵ atoms, between about 100 atoms and about 10⁵ atoms, between about 200 atoms and about 10⁵ atoms, between about 500 atoms and about 10⁵ atoms, between about 1000 atoms and about 10⁵ atoms, between about 2000 atoms and about 10⁵ atoms, between about 5000 atoms and about 10⁵ atoms, between about 10000 atoms and about 10⁵ atoms, greater than 70 but less than about 50000 atoms, between about 75 atoms and about 50000 atoms, between about 80 atoms and about 50000 atoms, between about 90 atoms and about 50000 atoms, between about 100 atoms and about 50000 atoms, between about 200 atoms and about 50000 atoms, between about 500 atoms and about 50000 atoms, between about 1000 atoms and about 50000 atoms, greater than 70 but less than about 20000 atoms, between about 75 atoms and about 20000 atoms, between about 80 atoms and about 20000 atoms, between about 90 atoms and about 20000 atoms, between about 100 atoms and about 20000 atoms, between about 200 atoms and about 20000 atoms, between about 500 atoms and about 20000 atoms, and/or between about 1000 atoms and about 20000 atoms.

As one skilled in the art will appreciate the nanoparticle aspects of the present invention relate not only to a single nanoparticle (i.e., a single nanoparticle core bonded to a single functional moiety) in isolation from other nanoparticles but also to collections of such nanoparticles (e.g., 2 or more of such nanoparticles, such as more than about 10, such as more than about 100, such as more than about 1000, such as more than about 10⁴, such as more than about 10⁵, such as more than about 10⁶, such as more than about 10⁷, such as more than about 10⁸, such as more than about 10⁹, more than about 10¹⁰, more than about 10¹¹, more than about 10¹², more than about 10¹³, more than about 10¹⁴, more than about 10¹⁵, more than about 10¹⁶, more than about 10¹⁷, more than about 10¹⁸, more than about 10¹⁹, more than about 10²⁰, and/or more than about 10²¹ of such nanoparticles.

Such collections can be substantially free of nanoparticles which contain more than a single functional moiety, for example, as in the case where the number of nanoparticles which contain more than a single functional moiety (“N²⁺”) divided by the number of nanoparticles which contain a single functional moiety (“N¹”), i.e., the quotient of N²⁺/N¹, is less than about 0.3, such as less than about 0.2, less than about 0.15, less than about 0.1, less than about 0.08, less than about 0.05, less than about 0.04, less than about 0.03, less than about 0.02, less than about 0.01, less than about 0.005, and/or less than about 0.001. As will be apparent from the discussion set forth above, a nanoparticle is to be construed as containing a single functional moiety if there is one point of attachment between the functional moiety and the nanoparticle core, irrespective of the number of functional groups which may be present on the functional moiety. For example, a nanoparticle which is made from a nanoparticle core having attached thereto one functional moiety bearing two carboxylic acid groups (or a carboxylic acid group and an amine group or any other combination of functional groups) is to be deemed to be a “nanoparticle which contains a single functional moiety”.

Such collections can also be substantially free of nanoparticles which contain no functional moieties, for example, as in the case where the number of nanoparticles which contain zero single functional moiety (“N⁰”) divided by the number of nanoparticles which contain a single functional moiety (“N¹”), i.e., the quotient of N⁰/N¹, is less than about 0.3, such as less than about 0.2, less than about 0.15, less than about 0.1, less than about 0.08, less than about 0.05, less than about 0.04, less than about 0.03, less than about 0.02, less than about 0.01, less than about 0.005, and/or less than about 0.001.

The nanoparticles of the present invention can be substantially pure. For the purposes of the present invention, a nanoparticle of the present invention is to be deemed to be “substantially pure” if it is substantially free from nanoparticles which contain more than a single functional moiety, for example, as in samples where the number of nanoparticles which contain more than a single functional moiety (“N²⁺”) divided by the number of nanoparticles which contain a single functional moiety (“N¹”), i.e., the quotient of N²⁺/N¹, is less than about 0.3, such as less than about 0.2, less than about 0.15, less than about 0.1, less than about 0.08, less than about 0.05, less than about 0.04, less than about 0.03, less than about 0.02, less than about 0.01, less than about 0.005, and/or less than about 0.001. Optionally, such substantially pure nanoparticles of the present invention can also be substantially free of nanoparticles which contain no functional moieties.

As discussed above, one aspect of the present invention relates to a nanoparticle which includes a nanoparticle core having a core diameter of greater than 5 nm and a single functional moiety bonded to the nanoparticle core. In one illustrative embodiment of this aspect of the present invention, the single functional moiety contains a nucleic acid molecule (e.g., a DNA molecule or an RNA molecule). In another illustrative embodiment, the single functional moiety does not contain a nucleic acid molecule (e.g., a DNA molecule or an RNA molecule). In still another illustrative embodiment, the single functional moiety contains a nucleic acid molecule comprising fewer than 50 nucleotides, as in the case where the single functional moiety contains a nucleic acid molecule comprising fewer than about 40 nucleotides. In yet another illustrative embodiment, the single functional moiety contains a nucleic acid molecule comprising 50 or more nucleotides. In yet other illustrative embodiments of this aspect of the present invention, the nanoparticle of the present invention further includes one or more non-functional moieties bonded to the nanoparticle core. In one such embodiment, the non-functional moieties are not phosphine moieties (e.g., a moiety which is not a trialkyl phosphine moiety and which is not a triphenylphosphine or other triarylphosphine moiety). In another such embodiment, the non-functional moieties are selected from the group consisting of alkylthio moieties, arylthio moieties, and combinations thereof.

As discussed above, another aspect of the present invention relates to a nanoparticle which includes a nanoparticle core having a core diameter of greater than 1.4 nm and a single functional moiety bonded to the nanoparticle core, wherein the single functional moiety does not contain a nucleic acid molecule comprising 100 or more nucleotides. In one illustrative embodiment of this aspect of the present invention, the nanoparticle core has a core diameter of greater than about 1.5 nm. In another illustrative embodiment, the nanoparticle core has a core diameter of greater than about 1.7 nm. In still another illustrative embodiment, the nanoparticle core has a core diameter of greater than about 1.8 nm. In yet another illustrative embodiment, the nanoparticle core has a core diameter of greater than about 2 nm. In still another illustrative embodiment, the nanoparticle is part of a collection of nanoparticles, and the collection of nanoparticles is substantially free from nanoparticles which contain more than a single functional moiety. In still another illustrative embodiment, the nanoparticle is part of a collection of nanoparticles, the collection of nanoparticles is substantially free from nanoparticles which contain more than a single functional moiety, and the collection of nanoparticles is substantially free from nanoparticles which contain no functional moieties. In yet another illustrative embodiment, the single functional moiety does not contain a nucleic acid molecule. In still other illustrative embodiments, the single functional moiety contains a nucleic acid molecule comprising fewer than 100 nucleotides, fewer than about 90 nucleotides, fewer than about 80 nucleotides, fewer than about 50 nucleotides, fewer than about 40 nucleotides, fewer than about 30 nucleotides, or fewer than about 20 nucleotides. In yet another illustrative embodiment, the single functional moiety contains a nucleic acid molecule comprising fewer than 100 nucleotides, and the nanoparticle core has a core diameter of greater than about 5 nm. In still other illustrative embodiments of this aspect of the present invention, the nanoparticle of the present invention further includes one or more non-functional moieties bonded to the nanoparticle core. In one such embodiment, the non-functional moieties are not phosphine moieties (e.g., a moiety which is not a trialkyl phosphine moiety and which is not a triphenylphosphine or other triarylphosphine moiety). In another such embodiment, the non-functional moieties are selected from the group consisting of alkylthio moieties, arylthio moieties, and combinations thereof. In yet another illustrative embodiment of this aspect of the present invention, the nanoparticle core includes metal atoms in which substantially all of the metal atoms are gold metal atoms; the nanoparticle core has a core diameter of greater than about 1.5 nm; the nanoparticle is part of a collection of nanoparticles, and the collection of nanoparticles is substantially free from nanoparticles which contain more than a single functional moiety; the single functional moiety does not contain a nucleic acid molecule; the nanoparticle further includes one or more non-functional moieties bonded to the nanoparticle core; the non-functional moieties are not non-functional phosphine moieties. In yet another illustrative embodiment of this aspect of the present invention, the nanoparticle core includes metal atoms in which substantially all of the metal atoms are gold metal atoms; the nanoparticle core has a core diameter of greater than about 1.5 nm; the nanoparticle is part of a collection of nanoparticles, and the collection of nanoparticles is substantially free from nanoparticles which contain more than a single functional moiety; the single functional moiety contains a nucleic acid molecule that includes fewer than 100 nucleotides; the nanoparticle further includes one or more non-functional moieties bonded to the nanoparticle core; and the non-functional moieties are not non-functional phosphine moieties. In yet another illustrative embodiment of this aspect of the present invention, the nanoparticle core includes metal atoms in which substantially all of the metal atoms are gold metal atoms; the nanoparticle core has a core diameter of greater than about 1.5 nm; the nanoparticle is part of a collection of nanoparticles, and the collection of nanoparticles is substantially free from nanoparticles which contain more than a single functional moiety; the single functional moiety contains a nucleic acid molecule that includes fewer than 100 nucleotides; the nanoparticle further includes one or more non-functional moieties bonded to the nanoparticle core; and the non-functional moieties are selected from the group consisting of non-functional alkylthio moieties, non-functional arylthio, and combinations thereof. In still another illustrative embodiment of this aspect of the present invention, the nanoparticle core includes metal atoms in which substantially all of the metal atoms are gold metal atoms; the nanoparticle core has a core diameter of greater than about 1.5 nm; the nanoparticle is part of a collection of nanoparticles, the collection of nanoparticles is substantially free from nanoparticles which contain more than a single functional moiety, and the collection of nanoparticles is substantially free from nanoparticles which contain no functional moieties; the single functional moiety contains a nucleic acid molecule which includes fewer than about 80 nucleotides; the nanoparticle further includes one or more non-functional moieties bonded to the nanoparticle core; and the non-functional moieties are selected from the group consisting of non-functional alkylthio moieties, non-functional arylthio, and combinations thereof.

The nanoparticles of the present invention can be prepared by any suitable method, such as the ones which are set forth below.

The present invention, in another aspect thereof, relates to a method for preparing a functionalized nanoparticle. The method includes providing a nanoparticle core; providing a functionalizing moiety which include a functional group and a reactive group, wherein the functional group is attached to a substrate surface and wherein the reactive group is capable of bonding to the nanoparticle core's surface; and contacting the nanoparticle core's surface with the functionalizing moiety under conditions effective to bind the functionalizing moiety's reactive group and the nanoparticle core's surface together. The functional group can then, optionally, be cleaved from the substrate surface.

Nanoparticle cores suitable for use in the method of the present invention include those described above. Illustratively, the nanoparticle core can include metal atoms, substantially all of which can be of the same type, for example, as in the case where substantially all of the metal atoms are selected from the group consisting of gold metal atoms, silver metal atoms, copper metal atoms, platinum metal atoms, palladium metal atoms, and combinations thereof. Other suitable cores include those that are made, for example, of a ceramic material or of other types of substantially inorganic materials. The nanoparticle core can be of any suitable size and shape, such as any of the sizes and shapes discussed hereinabove. For example, the nanoparticle core can have a diameter of greater than 1.4 nm; it can have a diameter of greater than about 1.4 nm; it can have a diameter of 1.4 nm; it can have a diameter of about 1.4 nm; it can have a diameter of less than 1.4 nm; it can have a diameter of less than about 1.4 nm; it can have a diameter of from about 0.5 to about 20 nm; and/or it can have a diameter of from about 1 to about 10 nm. The nanoparticle cores can be protected with one or more non-functional moieties, which non-functional moieties can form a monolayer on the nanoparticle core's surface. Suitable non-functional moieties include, for example, phosphine moieties, arylthio moieties, and alkylthio moieties, such as the ones described hereinabove. Examples of suitable alkylthio non-functional moieties include those having the formula —S—R where R represents an aryl-substituted or unsubstituted alkyl moiety (e.g., an aryl-substituted or unsubstituted alkyl moiety comprising a linear C1-C12 alkyl chain). Protected and unprotected nanoparticle cores can be obtained from commercial sources (e.g., from Ted Pella (Reading, Calif.)), or they prepared by any suitable method, such as those described in Brust et al., J. Chem. Soc., Chem. Commun., pp. 801ff, (1994); Hostetler et al., Langmuir, 14:17ff (1998); Handley, pp. 13-32 in Hayat, ed., Colloidal Gold: Principles, Methods, and Applications, New York: Academic Press (1989); Turkevich et al., Discussions of the Faraday Society, No. 11, pp. 55-74 (1951); Frens, Nature Phys. Sci., 241:20-22 (1973); Sutherland et al., Colloid. Interface Sci., 48:129-141 (1992); U.S. Pat. No. 5,609,907 to Natan; U.S. Pat. No. 4,313,734 to Leuvering; U.S. Pat. No. 6,262,129 to Murray et al.; Schmid, ed., Clusters and Colloids, Weinheim, Germany: VCH (1994); Massart, IEEE Transactions On Magnetics, 17:1247ff (1981); Ahmadi et al., Science, 272:1924ff (1996); Henglein et al., J. Phys. Chem., 99:14129ff (1995); Curtis et al., Angew. Chem. Int. Ed. Engl., 27:1530ff (1988); Weller, Angew. Chem. Int. Ed. Engl., 32:41ff (1993); Henglein, Top. Curr. Chem., 143:113ff (1988); Henglein, Chem. Rev., 89:1861ff (1989); Brus, Appl. Phys. A., 53:465ff (1991); Bahncmann, pp. 251ff in Pelizetti et al., eds., Photochemical Conversion and Storage of Solar Energy, Holland: Kluwer (1991); Wang et al., J. Phys. Chem., 95:525ff (1991); Olshaysky et al., J. Am. Chem. Soc., 112:9438ff (1990); and Ushida et al., J. Phys. Chem., 95:5382ff (1992), which are hereby incorporated by reference.

As discussed above, the method of the present invention further includes providing a functionalizing moiety which includes a functional group and a reactive group, wherein the functional group is attached to a substrate surface and wherein the reactive group is capable of bonding to the nanoparticle core's surface. Suitable functionalizing moieties include, for example, compounds which have the formula:

(RG)-Q-(FG)-(SS)

where RG is a reactive group, FG is a functional group, SS denotes a substrate surface, and Q is a linking moiety.

The nature of the reactive group (e.g., RG) will depend on the nature of the surface of the nanoparticle core to which it is to be bound. For example, where the nanoparticle core's surface comprises metal atoms, such as gold or silver (e.g., where the nanoparticle core is a gold nanoparticle core or a silver nanoparticle core), suitable reactive groups include thiol groups, such as SH groups or protected thiol groups (e.g., protected with an acetyl group or another alkylcarbonyl group or protected as a thioether, such as a t-butyl thioether, a trityl thioether, or a trialkylsilyl thioether).

The linking moiety, when employed, can be, for example, a substituted or unsubstituted alkylene moiety.

As used herein, “alkylene” refers to a bivalent alkyl group, where alkyl has the meaning given below.

Linear, branched, and cyclic alkylenes, as well as examples thereof, are defined in similar fashion with reference to their corresponding alkyl group. Examples of alkylenes include methylene (i.e., —CH₂—), eth-1,1-diyl (i.e., —CH(CH₃)—), eth-1,2-diyl (i.e., —CH₂CH₂—), prop-1,1-diyl (i.e. —CH(CH₂CH₃)—), prop-1,2-diyl (i.e., —CH₂—CH(CH₃)—), prop-1,3-diyl (i.e., —CH₂CH₂CH₂—), prop-2,2-diyl (e.g. —C(CH₃)₂—), cycloprop-1,1-diyl, cycloprop-1,2-diyl, cyclopent-1,1-diyl, cyclopent-1,2-diyl, cyclopent-1,3-diyl, cyclohex-1,1-diyl, cyclohex-1,2-diyl, cyclohex-1,3-diyl, cyclohex-1,4-diyl, but-2-en-1,1-diyl, cyclohex-1,3-diyl, but-2-en-1,4-diyl, but-2-en-1,2-diyl, but-2-en-1,3-diyl, but-2-en-2,3-diyl. Also included in the meaning of the term “alkylene” are groups having the formula —R′—R″—, where —R′ represents a linear or branched bivalent alkyl group and R″— represents a cycloalkyl group, such as moieties having the formula:

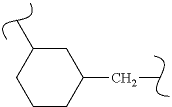

Also included in the meaning of the term “alkylene” are bivalent alkyl groups, such as those set forth above, in which one or more of the —CH₂— moieties is replaced with one or more heteroatoms (e.g., as in the case where the alkylene group has the formula —R′—X—R″—, —R′—X—, or —X—R″—, where X is O, S, Se, NH (which may be optionally substituted and —R′— and —R″— are the same or different are represent a linear or branched bivalent alkylene group. Also included in the meaning of the term “alkylene” are bivalent alkyl groups, such as those set forth above, in which adjacent carbons are involved in a double bond, as in the case where the alkylene group has the formula —R′—CH═CH—R″— (—R′— and —R″— being the same or different are representing a linear or branched bivalent alkylene group and one or both sp² hybridized carbons being optionally substituted).

As used herein, “alkyl” is meant to include linear alkyls, branched alkyls, and cycloalkyls, each of which can be substituted or unsubstituted. “Alkyl” is also meant to include lower linear alkyls (e.g., C1-C6 linear alkyls), such as methyl, ethyl, n-propyl, n-butyl, n-pentyl, and n-hexyl; lower branched alkyls (e.g., C3-C8 branched alkyls), such as isopropyl, t-butyl, 1-methylpropyl, 2-methylpropyl, 1-methylbutyl, 2-methylbutyl, 3-methylbutyl, 1,2-dimethylpropyl, 1,1-dimethylpropyl, 2,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 2-methyl-2-ethylpropyl, 2-methyl-1-ethylpropyl, and the like; and lower cycloalkyls (e.g., C3-C8 cycloalkyls), such as cyclopropyl, cyclobutyl, cyclopentyl, cyclohexyl, and the like. “Alkyl”, as use herein, is meant to include unsubstituted alkyls, such as those set forth above, in which no atoms other than carbon and hydrogen are present. “Alkyl”, as use herein, is also meant to include substituted alkyls. Suitable substituents include aryl groups (which may themselves be substituted), as in the case where the “alkyl” is a phenyl-substituted methyl group (e.g., a benzyl moiety). Other suitable substituents include heterocyclic rings (saturated or unsaturated and optionally substituted), hydroxy groups, alkoxy groups (which is meant to include aryloxy groups (e.g., phenoxy groups)), thiol groups, alkylthio groups, arylthio groups, amine groups (which is meant to include unsubstituted, monosubstituted, or disubstituted (e.g., with aryl or alkyl groups) amine groups), carboxylic acid groups (which is meant to include COOH groups as well as carboxylic acid derivatives, e.g., carboxylic acid esters, amides, etc.), phosphine groups, sulfonic acid groups, halogen atoms (e.g., Cl, Br, and I), and the like. Further, alkyl groups bearing one or more alkenyl or alkynyl substituents (e.g., a methyl group itself substituted with a prop-1-en-1-yl group to produce a but-2-en-1-yl substituent) is meant to be included in the meaning of “alkyl”.

The nature of the functionalizing moiety's functional group (e.g., FG), will be selected based on the nature of the substrate surface and the nature of the desired functionality.

For example, where the substrate surface comprises oxygen-containing groups (e.g., OH groups) or nitrogen-containing groups (e.g., NH₂ groups or other amine groups) and where it is desired that the functionalized nanoparticle be functionalized with a functional moiety comprising a carboxylic acid group, the functionalizing moiety's functional group can be a carboxyl group. Illustratively, suitable carboxyl groups include those having the formula —C(O)O— (e.g., such that the functionalizing moiety/substrate has the formula (RG)-Q-C(O)O-(SS)) and those having the formula —C(O)NR— (e.g., such that the functionalizing moiety/substrate has the formula (RG)-Q-C(O)NR-(SS)), where R represents, for example, a hydrogen atom, an alkyl group, etc.).

As a further example, where the substrate surface comprises carboxylic acid-containing groups (e.g., COOH groups or salts thereof) and where it is desired that the functionalized nanoparticle be functionalized with a functional moiety comprising a hydroxyl group or an amine group, the functionalizing moiety's functional group can be a carboxyl group. Illustratively, suitable carboxyl groups include those having the formula —OC(O)— (e.g., such that the functionalizing moiety/substrate has the formula (RG)-Q-OC(O)-(SS)) and those having the formula —N(R)C(O)— (e.g., such that the functionalizing moiety/substrate has the formula (RG)-Q-N(R)C(O)-(SS)), where R represents, for example, a hydrogen atom, an alkyl group, etc.).

In one illustrative embodiment of the method of the present invention, the functional group (e.g., FG) is a carboxyl group. In another illustrative embodiment of the method of the present invention, the reactive group (e.g., RG) is a thiol group. In yet another illustrative embodiment of the method of the present invention, the functional group (e.g., FG) is a carboxyl group, and the reactive group (e.g., RG) is a thiol group, such as in the case where the functionalizing moiety has the formula —O(O)C—R—SH and R represents a substituted or unsubstituted alkylene moiety.

As one skilled in the art will appreciate, where it is desired that the functionalized nanoparticle be functionalized with a functional moiety comprising a functional group other than a carboxylic acid group, an amine group, or a hydroxy group, such functionalized nanoparticles can be readily prepared using the method of the present invention and further converting the carboxylic acid group, amine group, or hydroxy group to the desired functional group. For example, where it is desired that the functionalized nanoparticle be functionalized with an aldehyde group, such a functionalized nanoparticle can be readily prepared using a functionalizing moiety having a formula (RG)-Q-C(O)O-(SS) in the method of the present invention to first prepare a functionalized nanoparticle (e.g., one having the formula (NC)-S-Q-C(O)O-(SS), where NC represents the nanoparticle core), cleaving the functional group from the substrate surface (e.g., by acid hydrolysis), and then converting the resulting nanoparticle (e.g., having the formula (NC)-S-Q-C(O)OH) to the aldehyde (e.g., having the formula (NC)-S-Q-C(O)H), for example, by converting the acid to the acid chloride and then reducing the acid chloride with a suitable reducing agent (e.g., Li(t-BuO)₃AlH. Methods for converting carboxylic acid groups, hydroxyl group, and amine groups to various other functional groups are well known to those skilled in the art and can be found, for example, in Carruthers, Some Modern Methods of Organic Synthesis, 3rd ed., New York: Cambridge University Press (1986); Smith, Organic Synthesis, 2nd ed., Boston: McGraw Hill (2002); Smith et al., Advanced Organic Chemistry, 5th ed., New York: J. Wiley Interscience (2001); Tietze et al., Reactions and Synthesis in the Organic Chemistry Laboratory, Mill Valley, Calif.: University Science Books (1989); Larock, Comprehensive Organic Transformations, 2nd ed., New York: Wiley VCH, (1999); Morrison et al., Organic Chemistry, 3rd ed., Boston: Allyn and Bacon, Inc. (1973); Kemp et al., Organic Chemistry, New York, Worth Publishers Inc. (1980); and House, Modern Synthetic Reactions, Menlo Park, Calif.: The Benjamin/Cummings Publishing Company (1972), which are hereby incorporated by reference.

Suitable substrate surfaces include, for example, the surface of any substrate. The surface can be planar, curved, or of any other shape. The substrate can be, for example, a solid material (e.g., where the external surface or surfaces of the material form the substrate surface) or a porous material (e.g., where the internal surfaces of the pores form at least part of the substrate surface), a sheet material, a bead, a mesh, etc. The size and shape of the substrate is not particularly critical to the practice of the present invention. For example, suitable substrates include beads, such as solid beads, which cannot be penetrated by the nanoparticle core (e.g., solid glass beads); and porous beads, which can be penetrated by the nanoparticle core (e.g., cross-linked polymer beads, such as cross-linked polystyrene beads). The beads can be of uniform size, or they can be of varying sizes; they can be of uniform shape (e.g., substantially spherical, ellipsoidal, polyhedral, flat, one-dimensional, etc.), or they can be of varying shapes. Illustratively, the beads can be substantially uniform in size and substantially spherical with a diameter of from about 1 μm to about 10 mm, such as from about 10 μm to about 1 mm, from about 20 μm to about 500 μm, from about 50 μm to about 200 μm, and/or about 100 μm. Other suitable substrates include, for example, paper, such as filter paper.

The arrangement of the functionalizing moieties on the substrate surface is not particularly critical. For example, the method can be carried out in the presence of a plurality of functionalizing moieties attached to surfaces (external and/or internal) of a plurality of beads. In such cases, the distribution of the functionalizing moieties on and/or in the beads is not particularly critical. For example, each bead can have substantially the same number of functionalizing moieties on the surface (external and/or internal) thereof, or some beads can have substantially fewer functionalizing moieties on their surfaces than others. On any particular bead, the functionalizing moieties can be arranged in any fashion; for example, the functionalizing moieties can be evenly distributed on the surface or surfaces (external and/or internal) of the bead, or they can be randomly distributed, with some functionalizing moieties being closer together than others.

As discussed above, the method of the present invention further includes contacting the nanoparticle core's surface with the functionalizing moiety. Such contacting can be carried out under conditions such that no more than one functionalizing moiety is bonded to each nanoparticle core. Alternatively, contacting the nanoparticle core's surface with the functionalizing moiety can be carried out under conditions such that more than one functionalizing moiety is bonded to each nanoparticle core. In the former case (i.e., where it is desired that no more than one functionalizing moiety be bonded to each nanoparticle core), the method of the present invention can be carried out in the presence of a plurality of functionalizing moieties which are separated by a distance D, where the distance D is substantially greater than the nanoparticle core's diameter. For the purposes of the present invention, a plurality of functionalizing moieties are to be deemed to be separated from each other by a distance D that is substantially greater than the nanoparticle core's diameter when at least 90% of the functionalizing moieties are separated from every other functionalizing moiety by a distance which is greater than 1 (e.g., at least about 1.1, at least about 1.2, at least about 1.3, at least about 1.4, at least about 1.5, at least about 1.8, at least about 2, at least about 2.5, at least about 3, at least about 5, at least about 10, at least about 15, at least about 20, and/or at least about 25) times the nanoparticle core's diameter.

Illustratively, where the nanoparticle core has a core diameter of about D′, suitable distances between functionalizing moieties can be achieved where the density of functionalizing moieties per unit surface area (nm²) is less than about Z, where Z is 1.25/(D′)² (i.e., the number 1.25 divided by the square of core diameter D′ (in nm)), such as in the case where the density of functionalizing moieties per unit surface area (nm²) is less than about 0.9Z, less than about 0.8Z, less than about 0.5Z, less than about 0.2Z, less than about 0.1Z, less than about 0.08Z, less than about 0.05Z, less than about 0.01Z, between about 0.02Z and Z, between about 0.05Z and Z, between about 0.08Z and Z, between about 0.1Z and Z, between about 0.2Z and Z, between about 0.5Z and Z, between about 0.8Z and Z, between about 0.01Z and Z, between about 0.01Z and about 0.9Z, between about 0.01Z and about 0.8Z, between about 0.01Z and about 0.5Z, between about 0.01Z and about 0.2Z, and/or between about 0.01Z and about 0.1Z. As further illustration, where the nanoparticle core has a core diameter of about 2 nm, suitable distances between functionalizing moieties can be achieved where the density of functionalizing moieties per unit surface area (nm²) is less than about 1.25, such as in the case where the density of functionalizing moieties per unit surface area (nm²) is less than about 1.125, less than about 1, less than about 0.625, less than about 0.25, less than about 0.125, less than about 0.0625, less than about 0.025, less than about 0.0125, between about 0.0125 and 1.25, between about 0.025 and 1.25, between about 0.0625 and 1.25, between about 0.1 and 1.25, between about 0.125 and 1.25, between about 0.25 and 1.25, between about 0.625 and 1.25, between about 1 and 1.25, between about 0.025 and about 1.125, between about 0.025 and about 1, between about 0.025 and about 0.625, between about 0.025 and about 0.5, between about 0.025 and about 0.125, and/or between about 0.025 and about 0.1.

In cases where a substrate is made of a porous material (e.g., beads that can be penetrated by the nanoparticle core, such as cross-linked polystyrene beads or other cross-linked polymer beads), suitable distances between functionalizing moieties (i.e., distances that are substantially greater than the nanoparticle core's diameter (D′)) can be achieved where the density of functionalizing moieties per unit volume (nm³) is less than about Z′, where Z′ is 1.9/(D′)² (i.e., the number 1.9 divided by the cube of core diameter D′ (in nm)), such as in the case where the density of functionalizing moieties per unit surface area (nm²) is less than about 0.9Z′, less than about 0.8Z′, less than about 0.5Z′, less than about 0.2Z′, less than about 0.1Z′, less than about 0.08Z′, less than about 0.05Z′, less than about 0.01Z′, between about 0.02Z′ and Z′, between about 0.05Z′ and Z′, between about 0.08Z′ and Z′, between about 0.1Z′ and Z′, between about 0.2Z′ and Z′, between about 0.5Z′ and Z′, between about 0.8Z′ and Z′, between about 0.01Z′ and Z′, between about 0.01Z′ and about 0.9Z′, between about 0.01Z′ and about 0.8Z′, between about 0.01Z′ and about 0.5Z′, between about 0.01Z′ and about 0.2Z′, and/or between about 0.01Z′ and about 0.1Z′. As further illustration, where the nanoparticle core has a core diameter of about 2.5 nm, suitable distances between functionalizing moieties can be achieved where the density of functionalizing moieties per unit volume (nm³) is less than about 0.12, such as in the case where the density of functionalizing moieties per unit surface area (nm²) is less than about 0.11, less than about 0.1, less than about 0.08, less than about 0.05, less than about 0.02, less than about 0.01, less than about 0.008, less than about 0.005, less than about 0.002, between about 0.002 and 0.12, between about 0.005 and 0.12, between about 0.008 and 0.12, between about 0.01 and 0.12, between about 0.02 and 0.12, between about 0.05 and 0.12, between about 0.08 and 0.12, between about 0.1 and 0.12, between about 0.002 and about 0.11, between about 0.002 and about 0.1, between about 0.002 and about 0.8, between about 0.002 and about 0.5, between about 0.002 and about 0.2, between about 0.002 and about 0.1, between about 0.002 and about 0.08, between about 0.002 and about 0.05, between about 0.002 and about 0.02, and/or between about 0.002 and about 0.01.

The functionalizing moiety can be prepared by any suitable method. One suitable method involves contacting a substrate bearing reactive groups on the surface thereof (e.g., polymeric beads bearing hydroxy groups, thiol groups, carboxylic acid groups, amine groups, etc.) with a compound bearing two functionalities, one of which is reactive with the reactive groups on the substrate's surface, and the other of which is either not reactive with the reactive groups on the substrate's surface or is protected from such reaction (e.g., using a suitable protecting group). The compound and the substrate are contacted under conditions effective for the substrate's reactive groups to react with the compound's reactive functionality to form a substrate-bound, optionally protected functionalizing moiety. Suitable conditions, such as solvent, use and selection of catalyst, reaction temperature, reaction time, etc., depend, for example, on the nature of the substrate, the nature of any other functional groups which may be present, the reactivity of the substrate's reactive groups and compound's reactive functionality, and the like. For example, where the substrate's reactive groups and compound's reactive functionality react via ester formation, suitable reaction conditions include those set forth in Fields, Methods in Enzymology. Volume 289: Solid Phase Peptide Synthesis, New York: Academic Press (1997) and in Fields et al., Int. J. Peptide Protein Res., 35:161ff (1990), which are hereby incorporated by reference. Where other reactions are involved, suitable reaction conditions can be readily ascertained, for example, by reference to Carruthers, Some Modern Methods of Organic Synthesis, 3rd ed., New York: Cambridge University Press (1986); Smith, Organic Synthesis, 2nd ed., Boston: McGraw Hill (2002); Smith et al., Advanced Organic Chemistry, 5th ed., New York: J. Wiley Interscience (2001); Tietze et al., Reactions and Synthesis in the Organic Chemistry Laboratory, Mill Valley, Calif.: University Science Books (1989); Larock, Comprehensive Organic Transformations, 2nd ed., New York: Wiley VCH, (1999); Morrison et al., Organic Chemistry, 3rd ed., Boston: Allyn and Bacon, Inc. (1973); Kemp et al., Organic Chemistry, New York, Worth Publishers Inc. (1980); and House, Modern Synthetic Reactions, Menlo Park, Calif.: The Benjamin/Cummings Publishing Company (1972), which are hereby incorporated by reference. Once the substrate-bound, optionally protected functionalizing moiety is formed, it can be used without further modification, or it can be further modified, for example, by deprotecting the protected functionalizing moiety to produce a substrate-bound functionalizing moiety. In one illustrative embodiment, the functionalizing moiety can be prepared by providing a substrate having hydroxyl groups on the surface thereof (e.g., having the formula HO-(SS), where SS represents the substrate surface) and contacting the substrate with a protected thiolalkylcarboxylic acid or a salt thereof (e.g., an alkylcarbonyl-protected HS—R—C(O)OH, such as a compound having the formula CH₃C(O)S—R—C(O)OH), where R represents a substituted or unsubstituted alkylene group) under conditions effective for the substrate's hydroxyl groups to react with the protected thiolalkylcarboxylic acid (or salt thereof). The resulting substrate-bound protected functionalizing moiety (e.g., a material having the formula CH₃C(O)S—R—C(O)—O-(SS), where SS represents the substrate surface) can then be deprotected (e.g., using ammonia in an appropriate solvent or solvent system, such as 3 M NH₃ in dioxane/water) to produce a substrate-bound functionalizing moiety, e.g., having the formula HS—R—C(O)—O-(SS).

Once the nanoparticle core and functionalizing moiety are provided, for example, as discussed above, the nanoparticle core's surface is contacted with the functionalizing moiety under conditions effective to bind the functionalizing moiety's reactive group and the nanoparticle core's surface together, such as conditions commonly employed in place exchange reactions. For example, in one suitable procedure, nanoparticle cores are suspended or dissolved in a suitable solvent, such as a chlorinated hydrocarbon (e.g., chloroform, methylene chloride, etc.). The resulting solution or suspension is then combined with (e.g., added to) the functionalizing moiety, which can be provided as a solid or as a suspension. The resulting mixture is then permitted to react, optionally with continuous or intermittent agitation (e.g., stirring, swirling, shaking and the like), for example, at a temperature of from about 10° C. to about the boiling point of the reaction solvent, such as from about 10° C. to about 60° C. and/or at about room temperature, for a period of time ranging from about 30 minutes to about 2 weeks, such as from about 3 hours to about one week and/or from about 6 hours to about 4 days. In one embodiment, the reaction is carried out (with or without agitation) at about room temperature for from about 2 days to about 4 days (such as for about 3 days), and then at from about 35° C. to about 55° C. (such as at from about 40° C. to about 50° C. and/or from about 45° C. to about 48° C.) for from about 4 hours to about 12 hours (e.g., for from about 6 hours to about 10 hours and/or for about 8 hours).

The resulting functionalized nanoparticles can then be isolated, for example, by filtration, centrifugation, etc., with optional washing, for example to remove any unreacted functionalizing moiety.

The resulting isolated functionalized nanoparticles can, optionally, be cleaved from the substrate. In cases where the functionalizing moiety's functional group is attached to the substrate surface via an ester linkage, cleavage can be carried out by using conventional ester hydrolysis conditions, such as by contacting the optionally isolated functionalized nanoparticles with trifluoroacetic acid in a suitable solvent (e.g., a chlorinated hydrocarbon, such as chloroform or methylene chloride), optionally with continuous or intermittent agitation (e.g., stirring, swirling, shaking, and the like), for example, at a temperature of from about 10° C. to about the boiling point of the reaction solvent, such as from about 10° C. to about 60° C. and/or at about room temperature, for a period of time ranging from about 10 minutes to about 1 week, such as from about 1 hour to about 1 day and/or from about 2 hours to about 3 hours. In cases where the functionalizing moiety's functional group is attached to the substrate surface via a linkage other than an ester linkage, suitable cleavage reaction conditions can be readily ascertained by those skilled in the art, for example, with reference to Carruthers, Some Modern Methods of Organic Synthesis, 3rd ed., New York: Cambridge University Press (1986); Smith, Organic Synthesis, 2nd ed., Boston: McGraw Hill (2002); Smith et al., Advanced Organic Chemistry, 5th ed., New York: J. Wiley Interscience (2001); Tietze et al., Reactions and Synthesis in the Organic Chemistry Laboratory, Mill Valley, Calif.: University Science Books (1989); Larock, Comprehensive Organic Transformations, 2nd ed., New York: Wiley VCH, (1999); Morrison et al., Organic Chemistry, 3rd ed., Boston: Allyn and Bacon, Inc. (1973); Kemp et al., Organic Chemistry, New York, Worth Publishers Inc. (1980); and House, Modern Synthetic Reactions, Menlo Park, Calif.: The Benjamin/Cummings Publishing Company (1972), which are hereby incorporated by reference.

In one illustrative embodiment of the method of the present invention, the nanoparticle core contains metal atoms; the nanoparticle core is protected with a monolayer of alkylthio moieties having the formula —S—R¹ on the nanoparticle core's surface; the functionalizing moiety has the formula —O(O)C—R²—S—H; each of R¹ and R² independently represents a substituted or unsubstituted alkylene moiety that includes a linear C1-C12 alkylene chain; and the linear C1-C12 alkylene chain of R² is longer than the linear C1-C12 alkylene chain of R¹.

The method of the present invention can, optionally, include other steps. Illustratively, the method of the present invention can further include reacting the functionalized nanoparticle with a reactive species to produce other functionalized nanoparticle. Examples of reactive species which can be used in such further steps include nucleic acid molecules, polypeptides (e.g., an antibody, a protein, etc.), and nanoparticles (e.g., a nanoparticle comprising a nanoparticle core having a core diameter of greater than 1.4 nm and a single functional moiety bonded to the nanoparticle core). Reaction of the functionalized nanoparticle with a reactive species can involve any functional group on the functionalized nanoparticle. Illustratively, in cases where the optional step of cleaving the functional group from the substrate surface, reaction of the functionalized nanoparticle with the reactive species can involve the functional group which is cleaved (e.g., liberated upon cleavage) from the substrate surface. Additionally or alternatively, in cases where the functional moiety contains a plurality of functional groups and not all (e.g., only one) of them are bonded to the nanoparticle core, reaction of the functionalized nanoparticle with the reactive species can involve functional groups that are not (and were not) bonded to the nanoparticle core. Thus, for example, the functionalized nanoparticle can be reacted, simultaneously or sequentially, with two or more reactive species, which two or more reactive species can be of the same type or of different types. Illustrative nanoparticles of the present invention (i.e., containing a single functional moiety bonded to a nanoparticle core, as described above), which can be prepared by such methods include, for example, those represented schematically by the formula (NC)-Q where Q represents a single functional moiety bonded to the nanoparticle core (NC), for example, as in the case where Q is represented schematically by one of the following formulae:

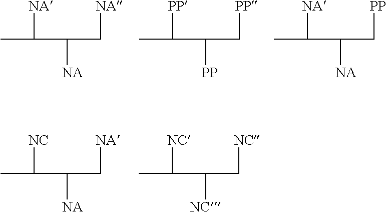

where NA, NA′, and NA″ are the same or different nucleic acid molecules; where PP, PP', and PP″ are the same or different polypeptides; and where NC′, NC″, and NC′″ are the same or different types of nanoparticle cores (e.g., based on core composition, core size, core shape, non-functional core protecting groups (if any), etc.). Illustratively, each of NC′, NC″, and NC′″ can be the same type of nanoparticle core (e.g., having the same type of nanoparticle core composition, size, shape, etc., and having the same type of non-functional core protecting groups) as one another, and/or each of NC′, NC″, and NC′″ can be the same type of nanoparticle core as NC.

Illustratively, such nanoparticles of the present invention wherein each of NC, NC′, NC″, and NC′″ represent the same type of nanoparticle core can be formed by reacting (i) nanoparticles of the present invention having the formula (NC)-R-Q′ where —R-Q′ represents the single functional moiety and comprises a carboxylic acid group (Q′) with (ii) an oligomer or a polymer bearing hydroxyl pendant groups (e.g., an oligomer or a polymer having the formula HO—CH₂— (CH₂)ₙ—[CH(OH)—(CH₂)ₙ]ₘ—H) to produce, for example, a nanoparticle of the present invention having the formula:

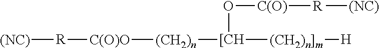

where n is an integer from 1 to 12, m is an integer from about 1 to about 10,000, and R is an alkylene moiety.

As further illustration, such nanoparticles of the present invention (i.e., wherein each of NC, NC′, NC″, and NC′″ represent the same type of nanoparticle core) can be formed by reacting, with one another, nanoparticles of the present invention having the formula (NC)-R(Q′)(Q″) where —R(Q′)(Q″) represents the single functional moiety comprising two functional groups (Q′ and Q″), such as a carboxylic acid group and an amine group. For example, —R(Q′)(Q″) can represent a single functional moiety having the formula —R—CH(COOH)—(CH₂)ₙ—NH₂), which, upon reaction with itself under conventional peptide bond formation conditions, can produce a nanoparticle of the present invention having the formula:

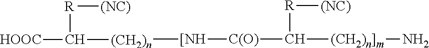

where n is an integer from 1 to 12 (e.g., 2, 3, or 4), m is an integer from about 1 to about 10,000, and R is an alkylene moiety. Alternatively, for example, —R(Q′)(Q″) can represent a single functional moiety having the formula:

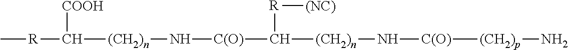

which, upon reaction with itself under conventional peptide bond formation conditions, can produce a nanoparticle of the present invention having the formula:

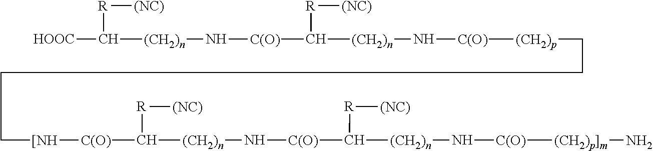

where n is an integer from 1 to 12 (e.g., 2, 3, or 4), p is an integer from 1 to 12 (e.g., 2, 3, or 4), m is an integer from about 1 to about 10,000, and R is an alkylene moiety.

It will be appreciated that the method of the present invention can be carried out using a nanoparticle core which has no functional moieties bonded thereto, in which case, when the nanoparticle core's surface is contacted with the functionalizing moiety under conditions such that no more than one functionalizing moiety binds to each nanoparticle core, the method produces a monofunctionalized nanoparticle (i.e., a nanoparticle that is functionalized with a single functional moiety).

It will be further appreciated that the method of the present invention can be carried out using a nanoparticle core which has a single functional moiety bonded thereto, in which case, when the nanoparticle core's surface is contacted with the functionalizing moiety under conditions such that no more than one functionalizing moiety binds to each nanoparticle core, the method produces a difunctionalized nanoparticle (i.e., a nanoparticle that is functionalized with a exactly two functional moieties).

Still further, it will be appreciated that the method of the present invention can be carried out using a nanoparticle core which has two functional moieties bonded thereto, in which case, when the nanoparticle core's surface is contacted with the functionalizing moiety under conditions such that no more than one functionalizing moiety binds to each nanoparticle core, the method produces a trifunctionalized nanoparticle (i.e., a nanoparticle that is functionalized with a exactly three functional moieties).

As one skilled in the art will also appreciate, the method of the present invention, when carried out under conditions such that no more than one functionalizing moiety binds to each nanoparticle core, can be used to add exactly one functional moiety to a nanoparticle core. Thus, stated generally, the method of the present invention can be carried out using a nanoparticle core which has N functional moieties bonded thereto, in which case, when the nanoparticle core's surface is contacted with the functionalizing moiety under conditions such that no more than one functionalizing moiety binds to each nanoparticle core, the method produces a nanoparticle that is functionalized with exactly N+1 functional moieties.

The present invention, in yet another aspect thereof, relates to a method for preparing a nanoparticle which includes a nanoparticle core having a core diameter of greater than 1.4 nm; and a single functional moiety bonded to the nanoparticle core, wherein the single functional moiety bonded to the nanoparticle core comprises a nucleic acid molecule. The method includes providing a nanoparticle which comprises a nanoparticle core having a core diameter of greater than 1.4 nm and a single functional moiety bonded to the nanoparticle core, wherein the single functional moiety is not a nucleic acid molecule and wherein the single functional moiety is capable of binding to a nucleic acid molecule. The method further includes contacting this nanoparticle with a nucleic acid molecule under conditions effective for the nucleic acid molecule to bind to the single functional moiety.

The invention described hereinabove has been set forth in the context of nanoparticles. As one skilled in the art will appreciate, the method of the present invention can be readily adapted to produce functionalized particles generally. For example, such particles can be microparticles (e.g., those having diameters ranging from about 1 μm to about 1000 μm and/or those having diameters between 1 μm and about 1000 μm, such as from about 1 μm to about 100 μm, from about 1 μm to about 50 μm, from about 1 μm to about 20 μm, from about 1 μm to about 10 μm, between 1 μm and about 100 μm, between 1 μm and about 50 μm, between 1 μm and about 20 μm, and/or between 1 μm and about μm), as well as particles having a diameter of greater than 1 mm, such as between 1 mm and about 10 mm.

Thus, stated more generally, the present invention also relates to a method for preparing a functionalized particle (e.g., a functionalized nanoparticle, a functionalized microparticle, etc.). The method includes, providing a particle core and providing a functionalizing moiety which includes a functional group and a reactive group, wherein the functional group is attached to a substrate surface and wherein the reactive group is capable of bonding to the particle core's surface. The method further includes contacting the particle core's surface with the functionalizing moiety under conditions effective to bind the functionalizing moiety's reactive group and the particle core's surface together. The functional group can then, optionally, be cleaved from the substrate surface. In cases where it is desired that no more than one functionalizing moiety be bonded to each particle core, the method of the present invention can be carried out in the presence of a plurality of functionalizing moieties which are separated by a distance D, where the distance D is substantially greater than the particle core's diameter, as in the case where at least 90% of the functionalizing moieties are separated from every other functionalizing moiety by a distance which is greater than 1 (e.g., at least about 1.1, at least about 1.2, at least about 1.3, at least about 1.4, at least about 1.5, at least about 1.8, at least about 2, at least about 2.5, at least about 3, at least about 5, at least about 10, at least about 15, at least about 20, and/or at least about 25) times the particle core's diameter.

Certain aspects of the present invention are further illustrated with the following examples. The examples also illustrate various applications in which the nanoparticles and methods of the present invention can be used.

### EXAMPLES

### Example 1

### Preparation of Monolayer-Protected Gold Nanoparticles with a Single Surface Functional Group

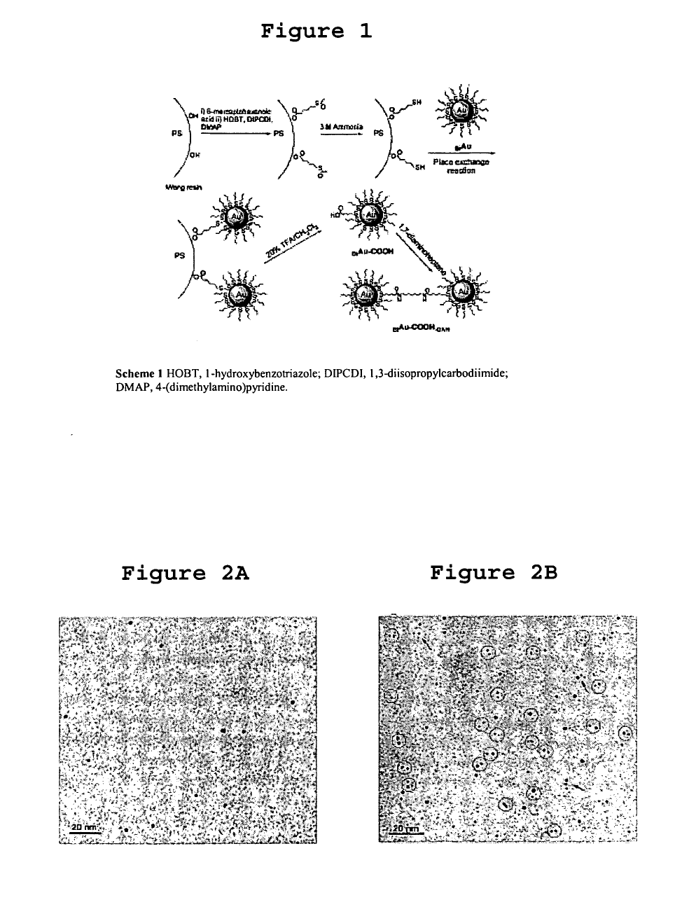

A solid phase synthesis technique was used to control the place exchange reaction and to prepare monolayer-protected gold nanoparticles with a single surface functional group, or an anisotropic distribution of functional groups. Solid phase synthesis is a synthetic strategy in which chemical reactions are conducted on a solid support such as a polymer resin. While this technique has been used in peptide and combinatorial library synthesis (Jung, ed., Combinatorial Peptide and Nonpeptide Libraries: A Handbook, New York: John Wiley & Sons (1997) (“Jung”) and Chaiken et al., eds., Molecular Diversity and Combinatorial Chemistry, Washington, D.C.: American Chemical Society (1996) (“Chaiken”), which are hereby incorporated by reference), it has never been applied in nanoparticle research. The synthetic strategy used in our study is outlined in Scheme 1, as set forth in FIG. 1. Briefly, a difunctional 6-mercaptohexanoic acid, with thiol group protected by an acetyl group (Svedhem et al., J. Org. Chem., 66:4494ff (2001) (“Svedhem”), which is hereby incorporated by reference), was attached to polystyrene Wang Resin through ester bond formation (Fields et al., Int. J. Peptide Protein Res., 35:161 (1990) (“Fields I”) and Fields, Methods in Enzymology Volume 289: Solid Phase Peptide Synthesis, New York: Academic Press (1997) (“Fields II”), which are hereby incorporated by reference). After the deprotection of the acetyl group, the thiol groups were allowed to undergo a place exchange reaction with butanethiolate-protected gold nanoparticles (“_{Bt}Au”), which were prepared according to the Shiffrin reaction with an average diameter of 2.8 nm (Brust et al., J. Chem. Soc., Chem. Commun., pp. 801ff, (1994), which is hereby incorporated by reference). The effective place exchange reaction between resin-bound thiol ligands and _{Bt}Au nanoparticles was clearly observed from the darkening of the resin beads after 12-24 hours of incubation of nanoparticles with resin beads in solution. After washing off any unexchanged nanoparticles, the resin-bound nanoparticles were cleaved from the resin using 20% trifluoroacetic acid (“TFA”) in dichloromethane. After washing and purification, pure nanoparticle product _{Bt}Au—COOH was obtained with a yield of around 50%. The obtained nanoparticles can be precipitated out and re-dissolved in organic solvents such as dichloromethane. Transmission electron microscopy (“TEM”) and X-ray diffraction (“XRD”) analysis show that the _{Bt}Au—COOH sample has the same average diameter of 2.8 nm as the _{Bt}Au nanoparticles, indicating that the structure of the nanoparticles remain intact during the solid phase synthesis.

It is believed that the number of functional groups attached to gold nanoparticles is controlled by the functional group density of the solid support. If the density of the free thiol groups attached to the Wang Resin is low enough that two adjacent thiol groups are relatively far away from each other as shown in Scheme 1, as set forth in FIG. 1, only one resin-bound thiol group will be attached to a gold nanoparticle by place exchange reaction. When the resin-bound nanoparticles are cleaved from the solid resin, nanoparticles with a single carboxylic group should be obtained as the major product. In our study, we chose Wang Resin with a functionality of 1.4-3.2 mmol per gram as the solid support. According to calculation, there would be less than one —SH per 20 nm³. Through solid phase synthesis gold nanoparticles with a single surface functional group should be obtained as the major product.

It is assumed that if there is only one carboxylic group present on the nanoparticle surface, when an aliphatic diamine is added to couple with the nanoparticles, one should see, by TEM analysis, the formation of nanoparticle pairs instead of trimers, tetramers, and larger aggregates formed from nanoparticles with more than one functional group. The coupling reaction was conducted according to the following conditions. Approximately 5 mg of dried _{Bt}Au—COOH was dissolved in 400 μL dichloromethane and 100 μL of dimethylformamide (“DMF”). To this solution, approximately 500 times equivalent N-hydroxysuccinimide (“NHS”) and 1-[3-(dimethylamino)propyl]-3-ethylcarbodiimide (“EDC”) in 100 μL DMF was added. The reaction mixture was heated at 40° C. for four hours followed by purification through a column of Sephadex LH-20 gel. To the activated nanoparticles about 100 times equivalent 1,7-diaminoheptane in a minimum of DMF was added to form the coupled nanoparticle product _{Bt}Au—COOH_{DAH} for TEM analysis.

FIGS. 2A and 2B are TEM images of _{Bt}Au—COOH nanoparticles and diamine-coupled _{Bt}Au—COOH_{DAH} nanoparticles, respectively. From these images, one can clearly see that the nanoparticles from the _{Bt}Au—COOH sample are randomly distributed while a large number of pairs appeared in the _{Bt}Au—COOH_{DAH} sample. The distance between the two nanoparticle cores in the pairs is around 1.0-1.5 nm, which corresponds roughly to the length of the organic ligand bridge between the two nanoparticles. By counting the number of nanoparticle pairs that appeared in several TEM images of this sample, it was estimated that about 60-70% of the solid phase synthesis product is single functional group-modified nano-particles. Analysis of some very diluted samples of _{Bt}Au—COOH_{DAH} indicates that the nanoparticle pairs observed in FIG. 2B are not due to a coincidental aggregation effect. At a closer look, a very small fraction (less than 5%) of nanoparticle trimers and aggregates are also present along with some individual nanoparticles (about 25%). The individual nanoparticles are likely non-functionalized nanoparticles or nanoparticles not coupled with diaminoheptane. The nanoparticle trimers and aggregates correspond to nanoparticles with more than one functional group. Additionally, UV-visible absorbance measurements show a 20 nm redshift of the surface plasmon resonance (“SPR”) band of the _{Bt}Au—COOH_{DAH} nanoparticles relative to the SPR band of the _{Bt}Au gold nanoparticles.

Theoretically, it may be possible to prepare gold nanoparticles with a single or other controlled numbers of functional groups through place exchange reaction in solution by using a very small ratio of the incoming thiol ligands relative to the replaced thiol ligand (Hostetler et al., Langmuir, 15:3782-2789 (1999) (“Hostetler”), which is hereby incorporated by reference). However, no in-depth study has ever been reported on the effectiveness of such an approach. To compare these two different approaches, we prepared two place exchange reaction products using 11-mercaptoundecanoic acid as the incoming ligand: one was targeting less than 5% exchange ratio (“_{Bt}Au—COOH_{5%}”) and another sample was aimed for 40% exchange ratio (“_{Bt}Au—COOH_{40%}”). The two place exchange reaction products were also coupled with 1,7-diaminoheptane for TEM analysis. It was noticed that, while the coupling product of _{Bt}Au—COOH_{5%} remained soluble in dichloromethane, the coupling product of _{Bt}Au—COOH_{40%} was insoluble in most organic solvents, indicating the formation of large aggregates due to multiple functional groups. For this reason, the TEM analysis of the 40% place exchange product was not obtained.

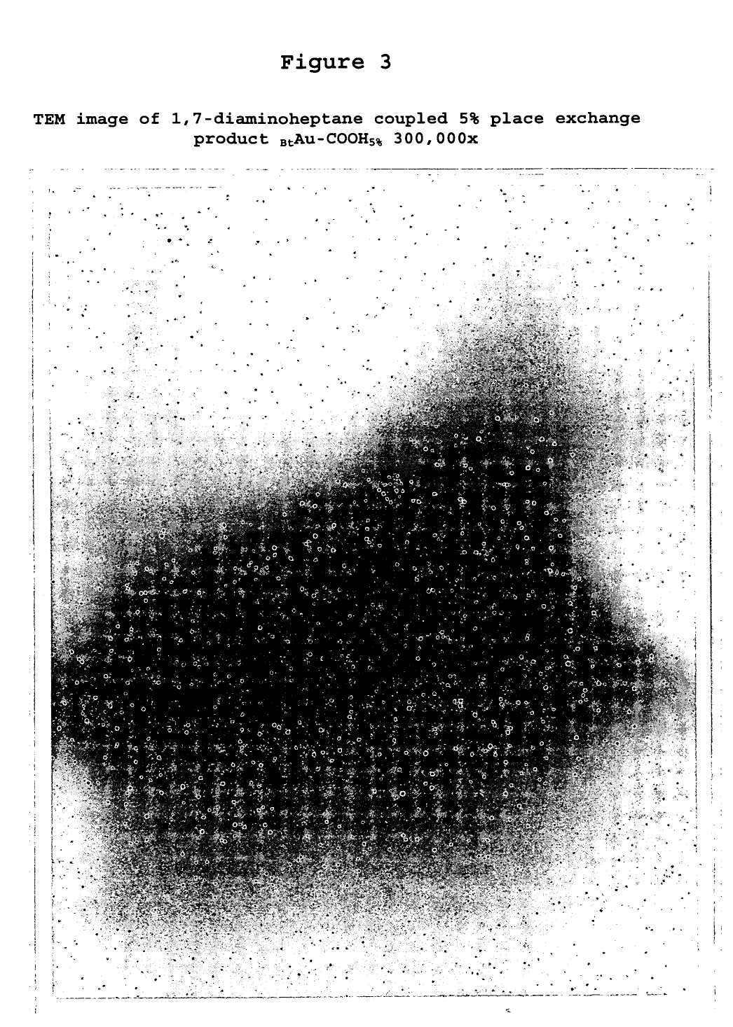

A TEM image of the 5% exchange product coupled by diaminoheptane is presented in FIG. 3 and shows the presence of mainly individual nanoparticles along with less than 6% nanoparticle dimers and trimers. This result indicates that, at least in some situations, the solid phase modification is a much better approach in preparing nanoparticles with controlled functionality. More particularly, in order to limit the number of functional groups attached to the nanoparticles during the place exchange reaction, the concentration of the incoming ligand has to be very low. As a result, the chance for the incoming ligands to collide with nanoparticles is extremely low, which inevitably leads to a low efficiency of ligand exchange. In contrast, in solid phase synthesis, the incoming ligands are immobilized on the polymer bead, and, by increasing the reaction temperature, nanoparticles have a much better chance of collision with the ligands to allow place exchange reaction to occur. More importantly, once the nanoparticle is attached to the beads, this nanoparticle will not react further with other ligands to allow attachment of multiple surface functional groups.

Experimental details with regard to the synthetic procedures described in this Example 1 are presented in Example 2.

### Example 2

### Experimental Details

Materials and Methods. All solvents and organic chemicals (ACS reagents) were purchased from Aldrich (Milwaukee, Wis.). 1% DVB crosslinked Wang resin with particle size 100-200 mesh and a hydroxyl group density of 1.4-3.2 mmol/g was obtained from Advanced ChemTech (Louisville, Ky.). This resin swells about 5-10 times (volume) in dichloromethane, according to the Quality Control Report from the company. We assumed a swellability of 10 times in dichloromethane since solid phase synthesis was conducted at elevated temperature of 40-45° C. Solid phase synthesis was conducted manually using a “home-made” system. The Sephadex gel used in size exclusion chromatography is a lipophilic dextrin gel LH-20 from Sigma with a lower separation limit of 7000 dalton molecular weight. The gel was pre-incubated in the corresponding eluent solvent for overnight prior to use. For transmission electron microscopic studies, approximately 1 μL of sample in the appropriate solvent was placed on a 300 mesh Formvar coated grid using an Eppendorf micropipette and immediately wicked off using filter paper. After allowing the sample to dry in air for 5-10 minutes, images were obtained using a JEOL 100CX Transmission Electron Microscope at 80 KeV. The TEM images were analyzed by NIH Image Software ImageJ v1.30. ¹H NMR spectra were obtained on a Varian Mercury 300 MHz spectrometer using a line-broadening factor of 1 Hz and relaxation delay of 5 seconds. X-Ray Diffraction analysis of nanoparticle samples was performed with a Philips automated diffractometer (PW3040 Multi-Purpose Diffractometer). Samples were ground into a fine powder, pipetted onto a quartz slide as a slurry in hexane, and air-dried. Crystalline phases were identified and analyzed using an MDI Jade Software v3.1. UV-visible absorbance measurements were acquired using an Agilent 8453 Spectrometer with a solvent mixture of 4:1 dichloromethane to methanol.

Solid Phase Synthesis of _{Bt}Au—COOH Nanoparticles. Acetyl-protected 6-mercaptohexanoic acid was prepared according to literature procedure (Svedhem, which is hereby incorporated by reference) and coupled to Wang resin in 1:2 mole ratio (thiol:hydroxyl group) using standard loading procedure for attaching amino acids to Wang resin (Fields I and Fields II, which are hereby incorporated by reference). After washing with DMF, dichloromethane and methanol followed by drying, the acetyl group was deprotected using ammonia solution (3 M in dioxane/water, 4/1, v/v) for 12-24 hours. Ellman's agent (5,5′-dithio-bisnitrobenzoic acid) was used to monitor the deprotection of the acetyl group visually. After the washing/drying cycle again, dichloromethane solution of _{Bt}Au nanoparticles (30 mg in 6 mL CH₂Cl₂, prepared according to the reported Schiffrin reaction using 1:1 mole ratio of HAuCl₄ and butanethiol at room temperature) was added and mixed with solid resin. The exchange reaction was allowed to proceed for four hours at 45° C. followed by twelve hours at room temperature with gentle shaking. During this time the beads turned dark black. After filtering and washing with warm dichloromethane, the dark beads were dried under vacuum and then suspended in dichloromethane (8 mL) for thirty minutes. The nanoparticles were then cleaved from the resin by adding 2.0 mL trifluoroacetic acid (equivalent to 20% TFA in CH₂Cl₂) and shaking the beads at room temperature for four hours. The dark nanoparticle solution was then collected and the nanoparticles were recovered by blowing off the solvent using a stream of N₂ gas.

The crude product was further purified by the following procedure. The dark solids were washed with petroleum ether 15-20 times with occasional sonication followed by centrifugation. After each cycle, the washing solution was tested for organic impurities by thin layer chromatography. The washing cycle was repeated until there was no longer UV active species appeared on the TLC plates and the washing solution became almost neutral. After drying, the nanoparticles were re-dissolved in a mixed solvent of 9:1 dichloromethane and methanol, and purified by size exclusion chromatography. The dark nanoparticle sample was collected in one portion. The sample was blown dry with N₂ stream and washed two additional times with petroleum ether and dried. The washing solution was found neutral (pH=7), and NMR analysis confirmed that the nanoparticle product was free of small organic molecules. The total yield of the purified _{Bt}Au—COOH nanoparticle is around 50%.

XRD analysis of the _{Bt}Au nanoparticles indicates that the nanoparticles have a core diameter of 1.84 nm relative to the 111 reflection and 1.57 nm relative to the 200 reflection. This core diameter corresponds to a nanoparticle diameter of 2.8 nm, in good agreement with the TEM analysis results. The different core sizes may indicate that the nanoparticles have more than one crystal structure.

Calculation of the Thiol Group Density on the Wang Resin During Solid Phase Synthesis. The thiol group density on the Wang Resin during solid phase synthesis was calculated with reference to Advanced ChemTech Handbook of Combinatorial & Solid Phase Organic Chemistry, 1998, Advanced ChemTech, Inc., page 100-101, which is hereby incorporated by reference. The Wang resin (1% DVB crosslinked polystyrene) used in this study has a functionality (—OH group) of 1.4-3.2 mmol/g and density of 1.05 grams/cm³. This resin swells about 5-10 times of its dry volume in CH₂Cl₂. A swellability of 10 times was used for the calculation, since the solid phase place exchange reaction was conducted at 40-45° C. According to these numbers, it is calculated that there is approximately one hydroxyl group per 10 nm³ in CH₂Cl₂ solution. Since only 0.5 equivalent of 6-mercaptohexanoic acid was loaded to Wang resin, there should be less than one thiol group per 20 nm³ attached to the resin for the solid phase place exchange reaction.

Coupling Reaction of _{Bt}Au—COOH Nanoparticles with 1,7-Diaminoheptane. To couple _{Bt}Au—COOH nanoparticles with 1,7-diaminoheptane, approximately 5 mg dried _{Bt}Au—COOH nanoparticles were dissolved in 400 μL dichloromethane and 100 μL DMF. To this solution, approximately 500 times equivalent of N-hydroxysuccinimide in 100 μL DMF was added followed by 500 times equivalent of 1-[3-(dimethylamino)propyl]-3-ethylcarbodiimide hydrochloride (“EDC”) in 100 μL DMF. The reaction mixture was incubated at 40° C. water bath for 4 hours followed by gentle shaking at room temperature for overnight. After blowing off most of the solvent, the reaction mixture was run through size exclusion chromatography to eliminate excessive organic reagents. The collected nanoparticle solution was concentrated by gentle nitrogen gas flow again. Typically, almost all the nanoparticles were re-collected after the activation reaction. The activated nanoparticles were re-dissolved in 500 μL dichloromethane and to the solution, approximately 100 times equivalent of 1,7-diaminoheptane was added and the reaction mixture was allowed to shake at room temperature for 24 hours. After blowing off the excessive solvent, the reaction mixture was purified by size exclusion chromatography to eliminate small organic reagent. The collected nanoparticle samples were subjected to TEM analysis by re-dissolving them in dichloromethane to the appropriate concentration.

Place Exchange Reaction to Prepare 5% and 40% 11-Mercaptoundecanoic Acid Substituted _{Bt}Au Nanoparticles: _{Bt}Au—COOH_{5%} and _{Bt}Au—COOH_{40%}. _{Bt}Au—COOH_{5%} and _{Bt}Au—COOH_{40%} were prepared in accordance with the general procedures set forth in Hostetler, which is hereby incorporated by reference.

_{Bt}Au—COOH_{5%} was prepared as follows. Approximately 70 mg _{Bt}Au nanoparticles were dissolved in 25 mL toluene. To this solution, ˜1.30 mg of 11-mercaptoundecanoic acid was added to give a 1:20 molar ratio of incoming thiol ligand versus the nanoparticle-bound thiolate. This reaction mixture, protected by N₂, was stirred between 48 and 96 hours for complete place exchange reaction to occur. After the reaction the solvent was removed using a rotary evaporator. The dark solid product was re-suspended in acetonitrile and washed by centrifugation for adequate times until no small organic species were found in the washing solution by TLC and NMR analysis.

The 40% place exchange product (_{Bt}Au—COOH_{40%} was obtained in a similar procedure except that a 1:2 molar ratio of 11-mercaptoundecanoic acid to the nanoparticle-bound thiolate was used for the place exchange reaction. The product was re-suspended, filtered and washed with adequate acetonitrile to eliminate any small organic residues.

Coupling Reaction of 5% and 40% Place Exchanged Product with 1,7-Diaminoheptane. The diamine coupling reaction was conducted using a similar condition as the coupling of _{Bt}Au—COOH nanoparticles by 1,7-diaminoheptane. After the coupling reaction, it was found that a large amount of precipitates appeared from the 40% place exchange sample. These precipitates were collected and rinsed with dichloromethane, mixed dichloromethane/methanol, and methanol, alternatively. The precipitates were found insoluble in most organic solvents. For this reason, TEM analysis was not conducted on this sample. The 5% place exchange sample remains as a solution after coupling reaction, although a very small amount of precipitates was also formed. After blowing off most of the solvent, the reaction mixture was purified by size exclusion chromatography to eliminate small organic molecules. The collected sample was then dissolved in dichloromethane to appropriate concentration for TEM analysis.

### Example 3

### Coupling Reaction of _{Bt}Au—COOH Nanoparticles with 1,7-Diaminoheptane

To couple _{Bt}Au—COOH nanoparticles with ethylenediamine (“EDA”), approximately 2 mg dried _{Bt}Au—COOH nanoparticles, prepared as described in Example 2, were dissolved in 50 μL DMF. To this solution, 1 μL of EDA/diisopropylcarbodiimide (“DIPCDI”) mixture solution (10 μL EDA plus 3 μL DIPCDI dissolved in 100 μL DMF) was added. The reaction mixture was shaken at room temperature for 2 hours. The nanoparticles precipitated out during the reaction. The solution was decanted carefully using a capillary tube. The nanoparticle precipitates were further washed with CH₂Cl₂ followed by methanol for 7-8 times accompanied by sonication and dried over vacuum to obtain product BtAu—COOH-EDA.

### Example 4

### General Procedure for the Synthesis of Single Functional Group-Modified Gold Nanoparticles (sf-AU-m)

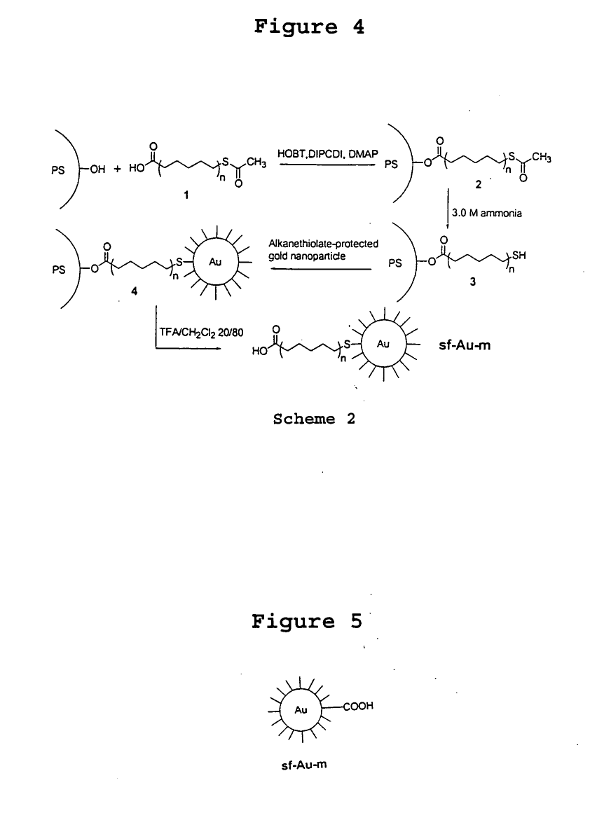

A general procedure for preparing gold nanoparticles modified with a single monofunctional group is illustrated in Scheme 2, as set forth in FIG. 4. Briefly, as shown in Scheme 2, an N-thioalkylcarboxylic acid protected with an acetyl group (1) is attached to a substrate, such as solid beads (e.g., Wang resin (“PS”) using standard ester bond coupling conditions. After deprotection of the acetyl group, the exposed groups from the resins (3) will then undergo place-exchange reaction with alkanethiolate-protected gold nanoparticles to form 4. Assuming that the thiol groups on the substrate (e.g., beads) are sufficiently far from each other, there will be only one thiol group from the resin to react with one gold nanoparticle. Accordingly, when cleaved from the substrate (e.g., resin), gold nanoparticles with a single copy of surface monofunctional group (sf-AU-m) will be obtained. It will be appreciated that, in Scheme 2, and elsewhere in this application, the short lines on the Au nanoparticle represent a plurality of alkanethiolates, such as an alkanethiolate monolayer. Gold nanoparticles with a single copy of surface monofunctional group (sf-AU-m) can also be represented using the notation set forth in FIG. 5, where the short lines on the Au nanoparticle represent a plurality of alkanethiolates, such as an alkanethiolate monolayer, and where the long line with the carboxylic acid group represents an n-mercaptoalkylcarboxylic acid group having an alkyl chain length that is longer than the alkyl chain length of the alkanethiolates.

### Example 5

### General Procedure for the Synthesis of Bifunctional Nanoparticles (sf-AU-b)

The focus of this example is the synthesis of gold nanoparticles with single copy of a bifunctional group (sf-Au-b). With a bifunctional group, these nanoparticles can be treated as molecules with two functional groups that can be coupled with another two molecules to synthesize larger molecules. The combined use of sf-Au-m and sf-Au-b particles will permit the synthesis of nanoparticle macromolecules and assemblies with crosslinked network structures, similar to a crosslinked polymer.

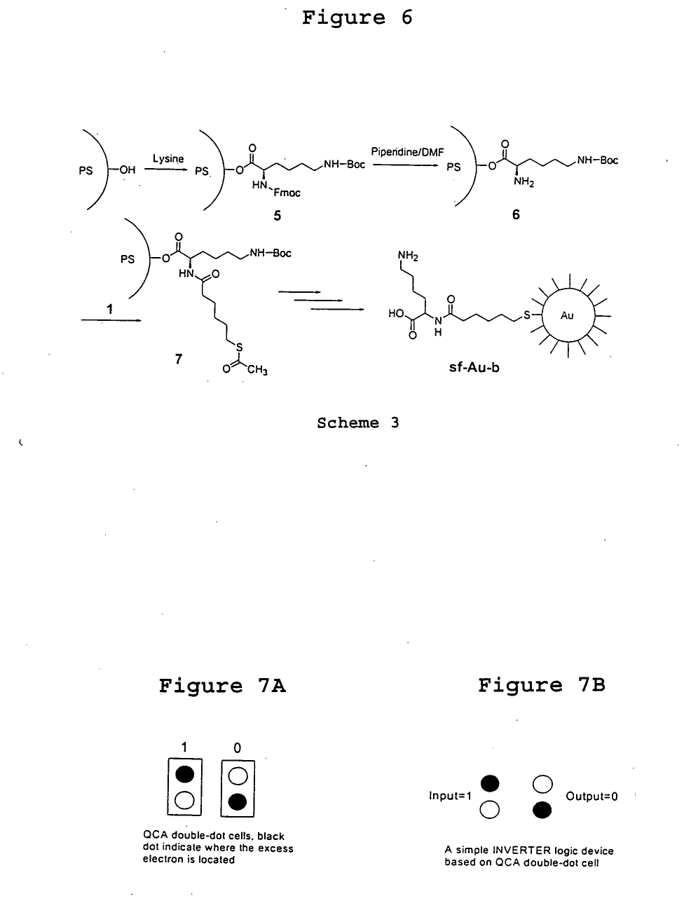

A solid phase synthetic method for the preparation of sf-Au-b nanoparticles with a pseudo amino acid structure is illustrated in Scheme 3, as set forth in FIG. 6. Solid phase peptide synthesis strategy based on Fmoc (fluorenylmethoxycarbonyl) chemistry is used for the synthesis (Fields I and Fields II, which are hereby incorporated by reference). Amino acid lysine will be first attached to the solid resins using reported loading procedure to form 5. Lysine has two amino groups, with the end one protected by a Fmoc group, and the side chain protected by a Boc (tert-butoxycarbonyl) group. The Fmoc group will be deprotected with piperidine/DMF (20/80), while the side chain amino group remains protected (the Boc group being stable under basic conditions), to form 6 (Fields I and Fields II, which are hereby incorporated by reference). The deprotected end amino group will react with acetyl-protected mercaptocarboxylic acid 1 through amide bond formation, leading to product 7. Once product 7 is formed, sf-Au-b is obtained by following the procedures used in the preparation of sf-Au-m. Briefly, the acetyl protecting group is removed, followed by place-exchange reaction with alkanethiolate-protected gold nanoparticles and subsequent cleavage to form gold nanoparticles with pseudo amino acid structures. During the acidic cleavage with trifluoroacetic acid, the Boc group on the side chain amino group will is deprotected. With one carboxylic group and one amino group, these sf-Au-b nanoparticles can be used as molecular building blocks in the synthesis of nanoparticle macromolecules, similar to peptide synthesis from amino acids.

### Example 6

### Optical Studies of sf-Au Nanoparticles

The sf-Au nanoparticles described herein can be used for single molecule study (Kneipp et al., Chem. Rev., 99:2957ff (1999); Campion et al., Chem. Soc. Rev., 27:241ff (1998); Keller et al., Anal. Chem., 74:316 Aff (2002); and Moerner et al., J. Phys. Chem. B., 106:910-927 (2002) (“Moerner”), which are hereby incorporated by reference). With only one molecule attached to the gold nanoparticle surface, any effect brought to the gold nanoparticle represents the interaction between a single molecule and the nanoparticle. The sf-Au nanoparticles provide uniquely appropriate models to study these interactions.

For example, it has been reported extensively that the optical properties of gold and silver nanoparticles such as UV-Vis absorption and fluorescence are significantly affected by nanoparticle-surface molecule interactions (Moerner; Franzen et al., J. Phys. Chem. A, 106:6533 (2002); Alvarez et al., J. Phys. Chem. B, 101:3706ff (1997); Larsson et al., J. Phys. Chem. B, 106:5931ff (2002); and Thomas et al., Langmuir, 18:3722ff (2002), which are hereby incorporated by reference). Surface enhanced Raman scattering (“SERS”) is a commonly observed phenomenon of gold nanoparticles (Corni et al., J. Chem. Phys., 116:1156ff (2002); Freeman et al., Science, 267:1629ff (1995); Doering et al., J. Phys. Chem. B, 106:311ff (2002); Nie et al., Science, 275:1102ff (1997); and Wei et al., Chem. Phys. Chem., 2:743ff (2001) (“Wei”), which are hereby incorporated by reference). However, the mechanism behind this phenomenon is still not clearly understood. It has been found that factors such as electromagnetic field interactions, chemical enhancement, surface active sites, and resonance Raman have important effects on SERS. Using the methods described herein, a single copy of an organic molecule can be attached to the sf-Au nanoparticles. Thus modified, such sf-Au nanoparticles can be used to study the chemical effect of a single molecule on SERS. For optical property studies, sf-Au nanoparticles around 10 nm (Wei, which is hereby incorporated by reference) can be synthesized and then modified with commercially available fluorescent dye molecules, such as fluorescein and rhodamine 6G. Thus modified, such sf-Au nanoparticles can be used to measure and analyze the effect of a single molecule on the UV-Vis absorption, fluorescence, FT-IR, and Raman spectra of sf-Au nanoparticles.

It has been reported that molecules that are bridged between two Ag nanoparticles exhibit a strong SERS effect, due to strong electromagnetic interactions (Michaels et al., J. Phys. Chem. B., 104:11965ff (2000), which is hereby incorporated by reference). Using the approach described herein, such structures can be synthesized by linking a dye molecule with two sf-Au nanoparticles, for example, by replacing EDA in Example 3 with a dye molecule containing two amino groups. The structure formed by linking a dye molecule with two sf-Au nanoparticles can be studied using Raman spectroscopy. From these optical property studies on single nanoparticle and particle pairs, further insights will be revealed on the single molecular interactions with gold nanoparticles and the mechanism of the SERS effect.

### Example 7

### Synthesis of Gold Nanoparticle Pair Macromolecules

Gold nanoparticles have been attracting much attention because of their application as quantum dots in quantum computer development. An example of quantum computing is quantum-dot cellular automata (“QCA”) devices, in which arrays of coupled quantum dots are used to implement logic functions (Tóth et al., Phys. Rev. A, 63:52315ff (2001); Snider et al., J. Appl. Phys., 85:4283ff (1999) (“Snider”); Amlani et al., Suprelattice Microstructure, 25:273ff (1999) (“Amlani”), which are hereby incorporated by reference). The principle of QCA is described briefly using a double-dot QCA cell as an example (FIG. 7A). When an excess electron is present in a QCA cell, this electron will occupy either of the two quantum dots, which represent the two logic states (0 or 1). In QCA devices, two QCA cells are placed close to each other so that electron tunneling between cells is not possible but so that the electrons in these two cells have a strong Coulomb interaction. In order to avoid the Coulomb repulsion between two electrons, the location of the excess electrons (0 or 1) in the output QCA cell is determined by the input cell (FIG. 7B). Information from input QCA cell is thus transferred to other cells. In fact, four-dot QCA cells are being used more commonly for QCA device development, although the principle of the double-dot and four-dot QCA cells is the same.

By appropriate design and arrangement of QCA cells, logic devices such as QCA lines, majority gates, and inverters have been developed (Snider, which is hereby incorporated by reference). The feasibility of this concept has been demonstrated on quantum cells made by electron beam lithography (Amlani, which is hereby incorporated by reference). Since electron beam lithography can only fabricate nanostructures around 100 nm, the quantum charging effect can only be observed at low temperatures around mK, and, therefore, such structures not applicable for room temperature applications. Recently double-dot devices have been reported using ˜60 nm Au disks (Junno et al., Appl. Phys. Lett., 80:667ff (2002), which is hereby incorporated by reference). Still, the size of the nanoparticle is too large to be used for room temperature applications. Extensive work has been reported on room temperature quantum charging effect of gold nanoparticles within a few nanometers range (Simon, Adv. Mater., 10:1487ff (1998); Chen et al., J. Am. Chem. Soc., 124:5280ff (2002); Chen, J. Am. Chem. Soc., 122:7420ff (2000) (“Chen II”); and Chen et al., Science, 280:2098ff (1998), which are hereby incorporated by reference).

Methods for the chemical synthesis of double-dot QCA cells using sf-Au nanoparticles are described hereinbelow. Also described is the further synthesis of QCA lines, which can function both as a wire and inverter, depending on the number of QCA cells in the line.

### Example 8

### Synthesis of Nanoparticle Pair: The Double-Dot OCA Cells

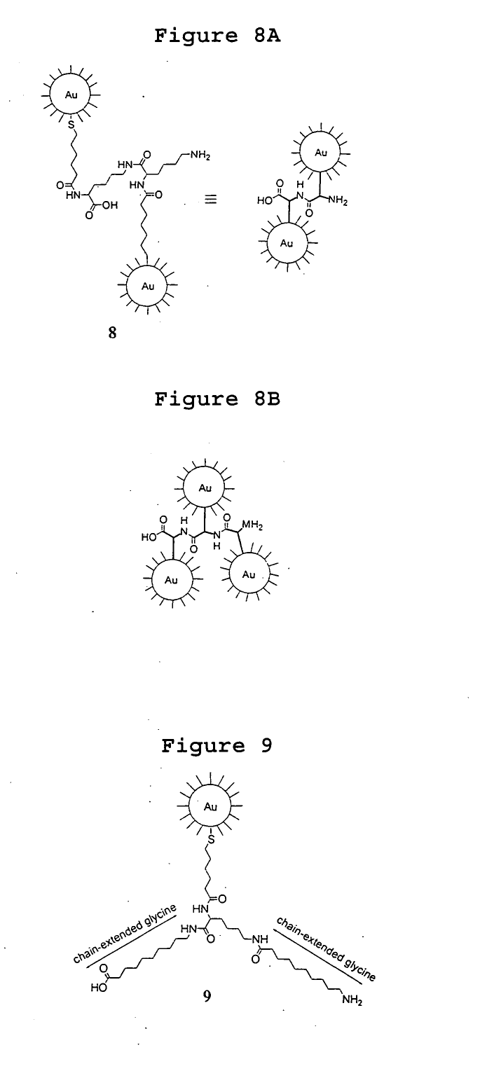

In this example, we described the synthesis of “double dots”, such as a nanoparticle pair illustrated in FIG. 8A. The synthetic method uses the sf-Au-b nanoparticles described hereinabove. The synthesis can be carried out by coupling two nanoparticles either through amide bond formation in solution or through solid phase synthesis, similar to the coupling of two amino acids to form a dipeptide. Using this approach, nanoparticle trimers (such as illustrated in FIG. 8B) can also be synthesized, for example, by coupling three sf-Au-b nanoparticles together. In situations where steric effect due to the gold nanoparticles may be a factor affecting the coupling of two or three nanoparticles (Templeton et al., J. Am. Chem. Soc., 120:1906ff (1998), which is hereby incorporated by reference), sf-Au-b nanoparticles with two chain-extended glycine amino acids (such as 9 in FIG. 9) can be synthesized and then used for nanoparticle pair synthesis. For example, a chain-extended glycine with 12 carbon atoms is around 2 nm long. With two glycine attached to side of the nanoparticle, the steric effect should be decreased significantly. Illustratively, for a gold nanoparticle with a 2 nm core and a hexanethiolate monolayer, the size of the nanoparticle is around 3.5 nm. To be used for QCA application, the distance between the two nanoparticles in the same cell should be close enough to allow electron tunneling to occur (Brousseau et al., J. Am. Chem. Soc., 120:7645ff (1998), which is hereby incorporated by reference). Such conditions can be met by synthesizing gold nanoparticle pairs using chain extended amino acid glycine with different number of carbon atoms as linker molecules.

### Example 9

### Synthesis of Nanoparticle Macromolecules with OCA Logic Gate Applications

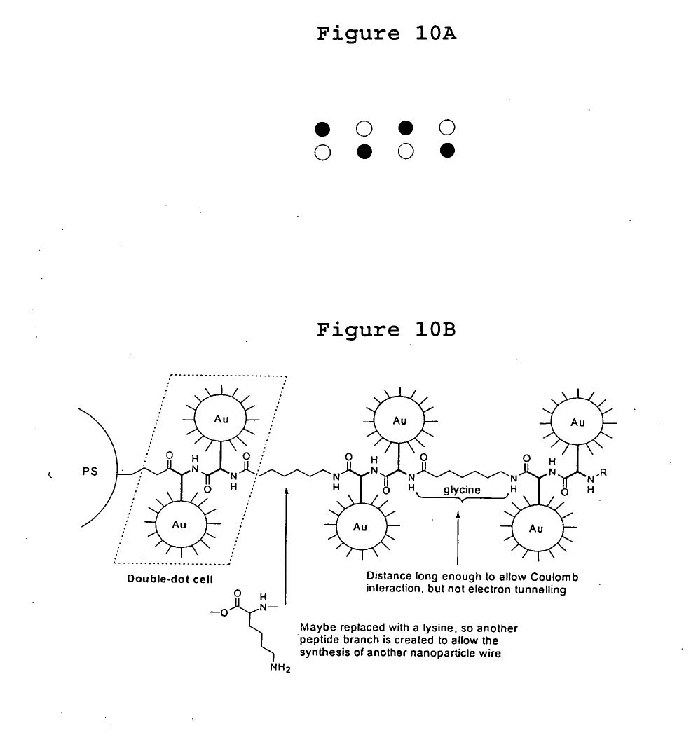

The gold nanoparticle pairs can serve as QCA cells (double dots). Accordingly, using the methods described herein, a nanoparticle line that mimics the structure of a QCA device can be prepared. One such QCA device is illustrated in FIG. 10A, and a nanoparticle line that mimics such a QCA device is illustrated in FIG. 10B. When the number of QCA cells in the line is even, the line functions as an inverter logic gate; and when the number is odd, the device is a QCA wire.

Solid phase synthesis can be used to synthesize such nanoparticle macromolecules. More particularly, gold nanoparticle pairs with a carboxylic acid and amino end groups are used as the building block for the solid phase synthesis. The coupling of one nanoparticle pair to the other is also through peptide bond formation. Between nanoparticle pairs, one or two chain-extended glycines can be coupled to the peptide backbone to increase the distance between two cells. Furthermore, if a lysine is inserted into the peptide backbone to replace glycine at a certain position, the side chain amino group can be used to react with one or more additional gold nanoparticle pairs to form a new nanoparticle branch, for example, as illustrated in FIG. 10B. By repeating this approach, large nanoparticle linear, planar, and three-dimensional macromolecules that mimic QCA devices can be synthesized chemically.

As one skilled in the art will appreciate, as the number of gold nanoparticle pairs attached to solid resin increases, the steric effect of gold nanoparticles may increase. In such cases, smaller components of nanoparticle macromolecules can be synthesized first in solid phase and then coupled together into larger macromolecules through solution phase synthesis. By employing solution phase synthesis, the steric effect can be decreased significantly by appropriate choice of solvent. Moreover, with longer reaction time, the reaction between the nanoparticles' functional groups can be promoted, thus promoting the formation of the desired nanoparticle macromolecules.

As discussed above, the distance between QCA cells needs to be controlled so that the two cells are far enough that electron tunneling between cells is not possible but close enough for Coulomb interactions to take place. This can be achieved by using chain-extended glycines as linker molecules for the synthesis of this linear nanoparticle macromolecule. QCA function can be further enhanced by increasing the rigidity of the double-dot cells, for example, by using dithiol compounds (Chen, Langmuir, 17:2878ff (2001) (“Chen I”), which is hereby incorporated by reference) to bridge the two nanoparticles.

### Example 10

### Self-Assembled Monolayer and Langmuir Monolayers of sf-Au Nanoparticles

The methods described herein can be used to prepare self-assembled monolayer of sf-Au nanoparticles on glass substrate and in Langmuir monolayers. The self-assembled monolayer and Langmuir-Blodgett films of alkanethiolate-protected gold nanoparticles have been investigated extensively on different substrates (Fendler, Chem. Mater., 8:1616ff (1996); Fendler, Chem. Mater., 13:3196ff (2001); Brust et al., Colloid Surface A, 202:175ff (2002); Chen et al., J. Phys. Chem. B, 106:1903ff (2002); Chen I; Chen, J. Phys. Chem., 104:663ff (2000); Chen II; Chen, Adv. Mater., 12:186ff (1986); and Chen, Langmuir 17:6664ff (2001), which are hereby incorporated by reference). It is believed that the hydrophilic carboxylic groups of the sf-Au nanoparticles tend to form non-covalent bond with glass substrate (or stay in aqueous phase in Langmuir monolayer), while the hydrophobic parts of the nanoparticle are exposed to the air. This can lead to the formation of two-dimensional self-assemblies with different network structures and optical properties compared to gold nanoparticles with multiple functional groups.

### Example 11

### Langmuir Monolayer Study of Peptide-Modified sf-Au Nanoparticles

Peptide-peptide interactions can be used to control the self-assembling of sf-Au nanoparticles in two-dimensions at the air-water interface of monolayers. It will be appreciated that peptides play a dominant role in not only biological functions, but they are also very useful in material research. The abundant structural varieties (including primary, secondary, and tertiary structures) make them promising molecules in supramolecular chemistry studies. Previous work has shown that peptide lipid molecules can form supramolecular assemblies at the air-water interface that have significant effect on the structures and properties of Langmuir monolayers (Berndt et al., J. Am. Chem. Soc., 117:9515ff (1995); Fields et al., Lett. Peptide Sci., 3:3ff (1996); Yu et al., J. Am. Chem. Soc., 118:12515ff (1996); Kunitake, Pure & Appl. Chem., 69:1999ff (1997); Cha et al., J. Am. Chem. Soc., 117:11833ff (1995); Cha et al., J. Am. Chem. Soc., 118:9545ff (1996); Cha et al., Chem. Lett., pp. 73ff (1996); Huo et al., Chem. Eu. J., 7:4796ff (2001); Huo et al., Angew. Chem. Int. Ed., 39:1854ff (2000) (“Huo I”); Huo et al., Chem. Comm., pp. 1601ff (1999); and Cao et al., Chem. Comm., pp. 806ff (2002), which are hereby incorporated by reference). Studies have shown that, due to the peptide-peptide interactions in the aqueous phase, the attached diacetylene hydrocarbon chains in the air phase are obliged to adopt different packing structures, leading to a completely different photopolymerization behavior of the diacetylene films (Huo I, which is hereby incorporated by reference).

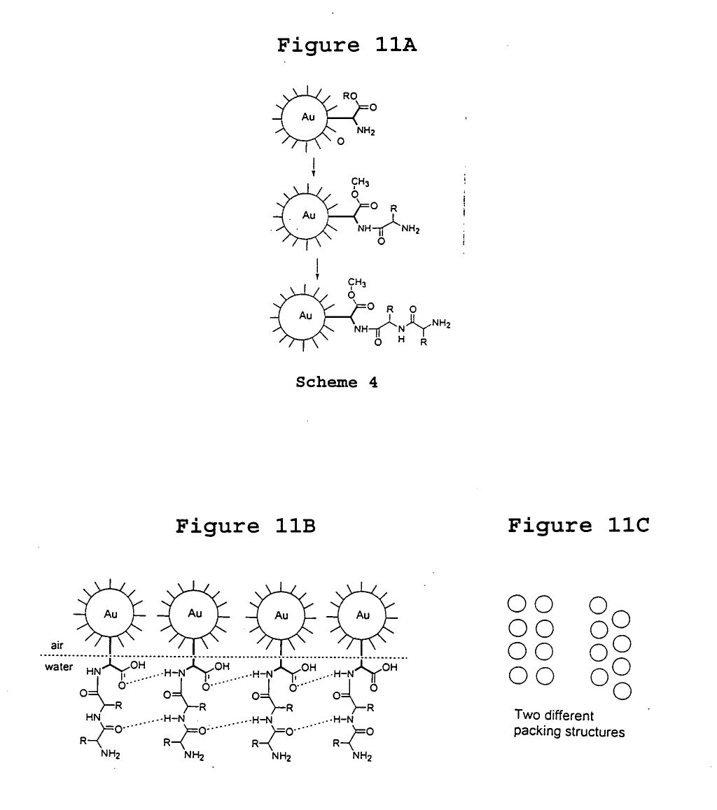

Using the gold nanoparticle-based solid phase synthesis technique described herein, sf-Au nanoparticles modified with peptides can be prepared. One such method is illustrated in Scheme 4, as set forth in FIG. 11A. In this case, the gold nanoparticles are used as solid support in the synthesis. After the coupling reaction of amino acids, there is no need to cleave off the product. The peptide-nanoparticle product will be spread at the air-water interface, with the peptide moieties staying in aqueous phase, and the hydrophobic parts of the nanoparticle oriented towards air, as illustrated in FIG. 11B. Upon compression, the peptide-peptide interaction such as hydrogen bonding between different nanoparticles will lead to different organization of nanoparticles in the monolayer. Based on previous reports, we believe that a very slight change in the peptide structures can result in a corresponding change in the two-dimensional network structures of the nanoparticle monolayer. Therefore, by changing the amino acid sequences of peptides, one can cause the gold nanoparticles to self-assemble into network structures having very fine differences, for example, as illustrated in FIG. 11C.

### Example 12

### Other Applications for Peptide-Modified sf-Au Nanoparticles

In addition to being useful in the formation of self-assembled structures, peptide-modified gold nanoparticles have other important applications.

For example, peptide-modified gold nanoparticles can be used in biochemistry and medicinal chemistry study. Illustratively, they can be used as solid support for peptide synthesis, for example, using the peptide synthesis methodologies reported in Templeton et al., J. Am. Chem. Soc., 120:4845ff (1998), which is hereby incorporated by reference. Since gold nanoparticles can be easily filtered through a funnel and separated from a reaction solution, peptide molecules or other organic compounds can be synthesized directly on gold nanoparticles.

Moreover, gold nanoparticles are known to be a good optical labeling material, for example, to detect bio-interactions, such as antibody-antigen binding, receptor-ligand recognition, and enzyme-substrate complexation. Without any further labeling steps, the peptide-modified gold nanoparticles described hereinabove can be used directly for activity screening. Furthermore, as contrasted with conventional gold-labeled peptides, the peptide-modified gold nanoparticles described hereinabove can have a single peptide attached to each gold nanoparticle. It is believed that the use of such peptide-modified gold nanoparticles as optical labels in bioassays and screening will permit more specific molecular recognition and bioactivities.

Although the invention has been described in detail for the purpose of illustration, it is understood that such detail is solely for that purpose, and variations can be made therein by those skilled in the art without departing from the spirit and scope of the invention which is defined by the following claims.

1. A nanoparticle comprising:
a nanoparticle core having a core diameter of greater than 5 nm; and
a single copy of a functional moiety bonded to the nanoparticle core, wherein said single functional moiety comprises at least one functional group.

2. A nanoparticle according to claim 1, wherein said nanoparticle core comprises metal atoms.

3. A nanoparticle according to claim 1, wherein said nanoparticle core comprises metal atoms in which substantially all of the metal atoms are of the same type.

4. A nanoparticle according to claim 1, wherein said nanoparticle core comprises metal atoms in which substantially all of the metal atoms are selected from the group consisting of gold metal atoms, silver metal atoms, copper metal atoms, platinum metal atoms, palladium metal atoms, and combinations thereof.

5. A nanoparticle according to claim 1, wherein said nanoparticle core comprises metal atoms in which substantially all of the metal atoms are gold metal atoms.

6. A nanoparticle according to claim 1, wherein said nanoparticle core has a core diameter of greater than about 5.5 nm.

7. A nanoparticle according to claim 1, wherein said nanoparticle core has a core diameter of greater than about 6 nm.

8. A nanoparticle according to claim 1, wherein said nanoparticle core has a core diameter of greater than 7 nm.

9. A nanoparticle according to claim 1, wherein said nanoparticle core has a core diameter of greater than about 10 nm.

10. A nanoparticle according to claim 1, wherein said nanoparticle is part of a collection of nanoparticles and wherein said collection of nanoparticles is substantially free from nanoparticles which contain more than a single functional moiety.

11. A nanoparticle according to claim 1, wherein said nanoparticle is part of a collection of nanoparticles and wherein said collection of nanoparticles is substantially free from nanoparticles which contain more than a single functional moiety and wherein said collection of nanoparticles is substantially free from nanoparticles which contain no functional moieties.

12. A nanoparticle according to claim 1, wherein said single functional moiety contains a nucleic acid molecule.

13. A nanoparticle according to claim 1, wherein said single functional moiety contains a nucleic acid molecule comprising fewer than 50 nucleotides.

14. A nanoparticle according to claim 1, wherein said single functional moiety contains a nucleic acid molecule comprising 50 or more nucleotides.

15. A nanoparticle according to claim 1, wherein said nanoparticle further comprises:
one or more non-functional moieties bonded to said nanoparticle core.

16. A nanoparticle according to claim 15, wherein said non-functional moieties are not non-functional phosphine moieties.

17. A nanoparticle according to claim 15, wherein said non-functional moieties are selected from the group consisting of non-functional alkylthio moieties, non-functional arylthio, and combinations thereof.

18. A nanoparticle comprising:
a nanoparticle core having a core diameter of greater than 1.4 nm; and
a single copy of a functional moiety bonded to the nanoparticle core, wherein said single functional moiety does not contain a nucleic acid molecule, and wherein said single functional moiety comprises at least one functional group.

19. A nanoparticle according to claim 18, wherein said nanoparticle core comprises metal atoms.

20. A nanoparticle according to claim 18, wherein said nanoparticle core comprises metal atoms in which substantially all of the metal atoms are of the same type.

21. A nanoparticle according to claim 18, wherein said nanoparticle core comprises metal atoms in which substantially all of the metal atoms are selected from the group consisting of gold metal atoms, silver metal atoms, copper metal atoms, platinum metal atoms, palladium metal atoms, and combinations thereof.

22. A nanoparticle according to claim 18, wherein said nanoparticle core comprises metal atoms in which substantially all of the metal atoms are gold metal atoms.

23. A nanoparticle according to claim 18, wherein said nanoparticle core has a core diameter of greater than about 1.5 nm.

24. A nanoparticle according to claim 18, wherein said nanoparticle core has a core diameter of greater than 1.7 nm.

25. A nanoparticle according to claim 18, wherein said nanoparticle core has a core diameter of greater than about 1.8 nm.

26. A nanoparticle according to claim 18, wherein said nanoparticle core has a core diameter of greater than about 2 nm.

27. A nanoparticle according to claim 18, wherein said nanoparticle is part of a collection of nanoparticles and wherein said collection of nanoparticles is substantially free from nanoparticles which contain more than a single functional moiety.

28. A nanoparticle according to claim 18, wherein said nanoparticle is part of a collection of nanoparticles and wherein said collection of nanoparticles is substantially free from nanoparticles which contain more than a single functional moiety and wherein said collection of nanoparticles is substantially free from nanoparticles which contain no functional moieties.

29.-37. (canceled)

38. A nanoparticle according to claim 18, wherein said nanoparticle further comprises:
one or more non-functional moieties bonded to said nanoparticle core.

39. A nanoparticle according to claim 38, wherein said non-functional moieties are not non-functional phosphine moieties.

40. A nanoparticle according to claim 38, wherein said non-functional moieties are selected from the group consisting of non-functional alkylthio moieties, non-functional arylthio, and combinations thereof.

41. A nanoparticle according to claim 18, wherein said nanoparticle core comprises metal atoms in which substantially all of the metal atoms are gold metal atoms; wherein said nanoparticle core has a core diameter of greater than 1.5 nm; wherein said nanoparticle is part of a collection of nanoparticles and wherein said collection of nanoparticles is substantially free from nanoparticles which contain more than a single functional moiety; wherein said nanoparticle further comprises one or more non-functional moieties bonded to said nanoparticle core; and wherein said non-functional moieties are not non-functional phosphine moieties.

42-73. (canceled)

74. A nanoparticle comprising:
a nanoparticle core having a core diameter of greater than 1.4 nm; and
a single copy of a functional moiety bonded to the nanoparticle core, wherein said single functional moiety contains a nucleic acid molecule comprising fewer than 100 nucleotides, and wherein said single functional moiety comprises at least one functional group.

75. A nanoparticle according to claim 74, wherein said nanoparticle core comprises metal atoms.

76. A nanoparticle according to claim 74, wherein said nanoparticle core comprises metal atoms in which substantially all of the metal atoms are of the same type.

77. A nanoparticle according to claim 74, wherein said nanoparticle core comprises metal atoms in which substantially all of the metal atoms are selected from the group consisting of gold metal atoms, silver metal atoms, copper metal atoms, platinum metal atoms, palladium metal atoms, and combinations thereof.

78. A nanoparticle according to claim 74, wherein said nanoparticle core comprises metal atoms in which substantially all of the metal atoms are gold metal atoms.

79. A nanoparticle according to claim 74, wherein said nanoparticle core has a core diameter of greater than about 1.5 nm.

80. A nanoparticle according to claim 74, wherein said nanoparticle core has a core diameter of greater than 1.7 nm.

81. A nanoparticle according to claim 74, wherein said nanoparticle core has a core diameter of greater than about 1.8 nm.

82. A nanoparticle according to claim 74, wherein said nanoparticle core has a core diameter of greater than about 2 nm.

83. A nanoparticle according to claim 74, wherein said nanoparticle is part of a collection of nanoparticles and wherein said collection of nanoparticles is substantially free from nanoparticles which contain more than a single functional moiety.

84. A nanoparticle according to claim 74, wherein said nanoparticle is part of a collection of nanoparticles and wherein said collection of nanoparticles is substantially free from nanoparticles which contain more than a single functional moiety and wherein said collection of nanoparticles is substantially free from nanoparticles which contain no functional moieties.

85. A nanoparticle according to claim 74, wherein said single functional moiety contains a nucleic acid molecule comprising fewer than about 90 nucleotides.

86. A nanoparticle according to claim 74, wherein said single functional moiety contains a nucleic acid molecule comprising fewer than about 80 nucleotides.

87. A nanoparticle according to claim 74, wherein said single functional moiety contains a nucleic acid molecule comprising fewer than about 50 nucleotides.

88. A nanoparticle according to claim 74, wherein said single functional moiety contains a nucleic acid molecule comprising fewer than about 40 nucleotides.

89. A nanoparticle according to claim 74, wherein said single functional moiety contains a nucleic acid molecule comprising fewer than about 30 nucleotides.

90. A nanoparticle according to claim 74, wherein said single functional moiety contains a nucleic acid molecule comprising fewer than about 20 nucleotides.

91. A nanoparticle according to claim 74, wherein said nanoparticle core has a core diameter of greater than about 5 nm.

92. A nanoparticle according to claim 74, wherein said nanoparticle further comprises:
one or more non-functional moieties bonded to said nanoparticle core.

93. A nanoparticle according to claim 92, wherein said non-functional moieties are not non-functional phosphine moieties.

94. A nanoparticle according to claim 92, wherein said non-functional moieties are selected from the group consisting of non-functional alkylthio moieties, non-functional arylthio, and combinations thereof.

95. A nanoparticle according to claim 74, wherein said nanoparticle core comprises metal atoms in which substantially all of the metal atoms are gold metal atoms; wherein said nanoparticle core has a core diameter of greater than about 1.5 nm; wherein said nanoparticle is part of a collection of nanoparticles and wherein said collection of nanoparticles is substantially free from nanoparticles which contain more than a single functional moiety; wherein said nanoparticle further comprises one or more non-functional moieties bonded to said nanoparticle core; and wherein said non-functional moieties are not non-functional phosphine moieties.

96. A nanoparticle according to claim 74, wherein said nanoparticle core comprises metal atoms in which substantially all of the metal atoms are gold metal atoms; wherein said nanoparticle core has a core diameter of greater than about 1.5 nm; wherein said nanoparticle is part of a collection of nanoparticles and wherein said collection of nanoparticles is substantially free from nanoparticles which contain more than a single functional moiety; wherein said nanoparticle further comprises one or more non-functional moieties bonded to said nanoparticle core; and wherein said non-functional moieties are selected from the group consisting of non-functional alkylthio moieties, non-functional arylthio, and combinations thereof.

97. A nanoparticle according to claim 74, wherein said nanoparticle core comprises metal atoms in which substantially all of the metal atoms are gold metal atoms; wherein said nanoparticle core has a core diameter of greater than about 1.5 nm; wherein said nanoparticle is part of a collection of nanoparticles, wherein said collection of nanoparticles is substantially free from nanoparticles which contain more than a single functional moiety, and wherein said collection of nanoparticles is substantially free from nanoparticles which contain no functional moieties; wherein said single functional moiety contains a nucleic acid molecule comprising fewer than about 80 nucleotides; wherein said nanoparticle further comprises one or more non-functional moieties bonded to said nanoparticle core; and wherein said non-functional moieties are selected from the group consisting of non-functional alkylthio moieties, non-functional arylthio, and combinations thereof.

98. A nanoparticle according to claim 1, wherein said functional group is monofunctional.

99. A nanoparticle according to claim 1, wherein said functional group is bifunctional.

100. A nanoparticle according to claim 18, wherein said functional group is monofunctional.

101. A nanoparticle according to claim 18, wherein said functional group is bifunctional.

102. A nanoparticle according to claim 74, wherein said functional group is monofunctional.

103. A nanoparticle according to claim 74, wherein said functional group is bifunctional.
